SEQUENCE LISTING (38 sequences)

SEQ ID NO: 1 - DNA, 1789 bp, Artificial Sequence
  cDNA
ggcacgagggttagtgttcgtaaaagttcttcttcctccttttttttctcggatccactaacgaatctcccagcgcaagtttgttcttctctgtaaatttctcaatctataaatactaaaccagcgaggaagtaatccatggctatggcgagtttgtatcggagatctcttccatctcctccggcgattgacttttcttctgccgagggaaagctaatcttcaatgaagctcttcagaaaggcaccatggaagggtttttcaggttgatttcttatttccaaacgcagtccgaacctgcgttttgtggtttggctagcctttctgtggtgttgaatgctctttctattgatcctggacgaaaatggaaagggccttggaggtggtttgatgaatcaatgctggattgctgcgagccactggaagtagtgaaggataagggaatttcatttggaaaagtcgtgtgtttggctcattgttcaggagcaaaagtggaagctttccgcacaaatcagagcaccattgataatttccgcaactttgtagtgaaatgcgcgacttctgataattgccatatgatctcaacatatcatagaggtgtgtttgagcagactgggtctggtcacttttcacctataggtggctataatgctgaaagagatatggctctgattcttgatgttgcccgtttcaagtatcctcctcactggattcctcttaaacttctttgggaagccatggacagcattgatgagacaacagggaaacgtagagggttcatgctcatatctagaccgcacagagaacctggattgctctatactctgagctgcaaggatgaaagctggatcagcatagcccagtatttgaaggaagatgttcctcgtcttgtaagttcagagaatgtagattctgtggaaaaaatcgtatcagttgtgttcaattcacttccctcaaaactcaaccaattcatcagatgggtggctgaggtcagaataacagaagacacaaacaaaaatctcagcgccgaggagaaatcgaggctgaagttaaagcaagtggtgctgaaagaagtgcaggaaactgaactgttcaaacacgtcagtaagtatttgtcctcagtgggttacgaggacagtctggcatatgcagctgcaaaggcttgttgccaaggagctgaaatcttgtcgggaacctcgtcaaaagagttctgttgtcgggaaacttgtgtgaaatgcgtcaaaggtcctgaagaggcagaaggcacggtggtgactggagttgtggtgcatgacgggagtgaacaaaaaattgatcttttggtgccatcgacccaaacagactgtgaatgtggttcggagaagaactatccatcaggaaacgatgtgttcactgtacttatgttggctttacctgcacagacatggtcagggatcaaagaccaagcttttatgcaagaaatgaagcagctcatttccatggcttccctcccaactatgcttcaagaagaggtattgcatcttcgacgtcaacttcagctgttaaaacgatgtcaagagaacaaggaagaggaagatctcgttgctcctgccttttgattcttctacccaaattcacactcttcttccccaatcgaatcccggtttttttaatataaaaccgtaattgtaagagagtattttattttccgtatgatattcaaactctatttgcagtgagagagatctgtatcctatataataataaagttataaaaccattatcatcccaaaaaaaaaaaaaaaaaaaaaa

SEQ ID NO: 2 - protein, 485 aa, Artificial Sequence
  phytochelatin synthase 1 ecoded by cDNA, clone #8
MAMASLYRRSLPSPPAIDFSSAEGKLIFNEALQKGTMEGFFRLISYFQTQSEPAFCGLASLSVVLNALSIDPGRKWKGPWRWFDESMLDCCEPLEVVKDKGISFGKVVCLAHCSGAKVEAFRTNQSTIDNFRNFVVKCATSDNCHMISTYHRGVFEQTGSGHFSPIGGYNAERDMALILDVARFKYPPHWIPLKLLWEAMDSIDETTGKRRGFMLISRPHREPGLLYTLSCKDESWISIAQYLKEDVPRLVSSENVDSVEKIVSVVFNSLPSKLNQFIRWVAEVRITEDTNKNLSAEEKSRLKLKQVVLKEVQETELFKHVSKYLSSVGYEDSLAYAAAKACCQGAEILSGTSSKEFCCRETCVKCVKGPEEAEGTVVTGVVVHDGSEQKIDLLVPSTQTDCECGSEKNYPSGNDVFTVLMLALPAQTWSGIKDQAFMQEMKQLISMASLPTMLQEEVLHLRRQLQLLKRCQENKEEEDLVAPAF

SEQ ID NO: 3 - DNA, 1311 bp, Artificial Sequence
  cDNA, clone #71
  misc_feature 463, 480: n = A,T,C or G
gaaaagccaagagaaggtgatgttgatgagaccagctagtaaaaccagtctgaccatcttcactctggttgttgtggtgaaaagaagcaagagagtgtaaagcttgtgaaagatagctgctgcggtgagaaaagtaggaaaccagagggagatatggcttcactgagctcatgcaagaagtctaacaatgacatgaaaatgaaaggtggttcaagttgttgtgctagtaaaaatgagaagctgaaggaagcagtagtagcaaagagctgctgtgaagacaaggagaaaacagagggaaatgttgagatgcagattccaaatttggagaaagggtcgcagaaaaaggttggtgaaacctgcaaatcaagctgttgtggagataaagagaaggctaaggaaacacgtttgttgcttgctagtgaggatccatcttatctggagaaggaagaaaggccaaactacntgaagctaacattgtgnccagtgaaacagagctgccatgagaaggcaagtctggacattgaaactggagttacttgtgatctcaagttggtctgctgcggaaacatagaagtgggagagcaatctgatcttgagaaaggcatgaagttaaagggtgaaggacaatgcaagtctgactgctgcggtgatgaaatacctctagcttctgaggaagacagtgtggattgctcctccggatgctgcggaaacaaggaggaattgacacaaatctgtcatgagaagacatgtctggacattgtaagttgtgattccaagttggtttgctgtggagaaacagaagtggaagtgagagagcaatgtgatctcaagaagggtctgcagataaagaatgaaggacaatgcaagtctgttcgttgcggtgatgaaaagaaaacagaggagataactgaagagacggacaatctgaaaagtgaaagtggtgatgattgcaaatctctttgttgtggaactggtttgaagcaagaagggtcttctagtttggtcaatgttgtggtggagagtggtgaatccgggtcaagctgttgcagcaaggagggagagatagtgaaagtctctagccaaagctgttgcgcaagtccaagtgatgtggtgttatctgacttggaagtcaagaaactagagatttgttgcaaagcgaagaagactccagaggaggttcgtggatctaaatgtaaggaaacagagaagcgtcaccacgttggtaaaagctgttgcaggagttatgcaaaagagtattgcagccacaggcatcaccaccaccaccaccaccaccatgttggggctgcttga

SEQ ID NO: 4 - protein, 1187 aa, Thlaspi caerulescens
  protein encoded by cDNA, clone #71
MALQKEIKNKEEDKKTKKKQRKSYFDVLGICCTSEIPLIENILKSLDGVKEYTVIVSPRTVIVVHDSLLISPFQIAKALNQARLEANVKVNGETSFKNKWPSPFAVVSGIFLLLSFLKFVYPPLRWLAVVGVAAGIYPILAKAVASIRRLRVDINILVIITVAATLAMQDYMEAAAVVFLFTIADWLETRTSYKANSVMQSLMSLAPQKAVIAETGEEVEVDEVQLNTIIAVKAGETIPIDGIVVDGNCEVDEKTLTGEAFPVPKQRDSTVLAGTMNLNGYISVNTTALASDCVVAKMAKLVEEAQGSKTKSQRLIDKCSQYYTPAIIIISAGFAIVPAIMKVHNLNHWFHLALVVLVSACPSGLILSTPVATFCALTKAATSGLLIKSADYLDTLSKIKIAAFDKTGTITRGEFIVIEFKSLSRDISLRSLLYWVSSVESKSSHPMAATIVDYAKSVSVEPRSEEVEDYQNFPGEGIYGKIDGNNVYIGNKRIASRAGCSTVPEIEVDTKKGKTVGYVYVGERLAGVFNLSDACRSGVAQAMKELKDLGIKTAMLTGDNQDSAMQAQEQLGNALDVVHGELLPEDKSKIIQEFKKEGPTCMVGDGVNDAPALANADIGISMGISGSALTTQTGHIILMSNDIRRIPQAIKLARRAQRKVLQNVFISITLKVGILVLAFAGHPLIWAAVLTDVGTCLIVILNSMLLLREKDKSKIKKCYRKKVEGGDDQGLDLEAGLLSKSQCNSGCCGDKKSQEKVMLMRPASKTSSDHLHSGCCGEKKQESVKLVKDSCCGEKSRKPEGDMASLSSCKKSNNDMKMKGGSSCCASKNEKLKEAVVAKSCCEDKEKTEGNVEMQIPNLEKKGSQKKVGETCKSSCCGDKEKAKETRLLLASEDPSYLEKEERQTTEANIVTVKQSCHEKASLDIETGVTCDLKLVCCGNIEVGEQSDLEKGMKLKGEGQCKSDCCGDEIPLASEEDSVDCSSGCCGNKEELTQICHEKTCLDIVSCDSKLVCCGETEVEVREQCDLKKGLQIKNEGQCKSVRCGDEKKTEEITEETENLKSESGDDCKSLCCGTGLKQEGSSSLVNVVVESGESGSSCCSKEGEIVKVSSQSCCASPSDVVLSDLEVKKLEICCKAKKTPEEVRGSKCKETEKRHHVGKSCCRSYAKEYCSHRHHHHHHHHHVGAA

SEQ ID NO: 5 - DNA, 608 bp, Artificial Sequence
  cDNA, clone #10
ggcacgaggcgaacatacacaagaactaaaacaatctttcaagcttttttcttctaaaaaaaccaatcatgtcggacaagtgcggaagctgcgactgttgggcacgaggcgaacatacacaagaactaaaacaatctttcaagcttttttcttctaaaaaaaccaatcatgtcggacaagtgcggaagctgcgactgttgtgacaagacccagtgcgtcacgaagagtaccagctacaccttggacatggtcgagactcaggagagctacaaggaggccatgaacatggacgttggtgcagaagagaacgggtgcaaatgcatgtgcggctctacctgcagctgcgtcaacggcacttgcagccccaactaaaagaaaaggctcctaaagaccttaaaacagggccatttctcttttcctccttttatcaaaatgtaatatgaataaaagtagatgtgagccacatctctctctctcttattatatgtaattcagactctctactatggcgtgatgtaattggtttatggccccttatcctctaatatacatcatcttatgatctaaaaaaaaaaaaaaaaaaaaaaaaaaaaaaaaaaaaaaaaaaa

SEQ ID NO: 6 - protein, 67 aa, Artificial Sequence
  protein encoded by cDNA, clone #10
MSDKCGSCDCCDKTQCVTKSTSYTLDMVETQESYKEAMNMDVGAEENGCKCMCGSTCSCVNGTCSPN

SEQ ID NO: 7 - DNA, 484 bp, Artificial Sequence
  cDNA, clone #51
ggcacgaggagaactcgaacatacacaagaactaaaacaatctttcaagcttttttcttctaaaaaaaccaatcatgactgacaagtgcggaagctgcgactgtgctgacaagacccagtccgtcaagaagagtaccagctacaccttggacatggtcgagactcaggagagctacaaggaggacatgaacatggacgttgttgcagaagagaacgggtgcaaatgcaagtgcggctctacctgcagctgcctcaactgcacttgcggcccaaactaaaaaaaggaccttaaaaaaggggccatttctagtttcatcctttgatcaaaatgtaatatgaataaaagttgatgtgagccacatctctctcttattaaaaatgtaattcagactcttcactatggcgtgatgtaaattagtttatggccccttatcctctaatatacatcatcttattatctattaaaaaaaaaaaaaaaaaaaaa

SEQ ID NO: 8 - protein, 67 aa, Artificial Sequence
  protein encoded by cDNA, clone #51
MTDKCGSCDCADKTQSVKKSTSYTLDMVETQESYKEDMNMDVVAEENGCKCKCGSTCSCLNCTCGPN

SEQ ID NO: 9 - DNA, 495 bp, Artificial Sequence
  cDNA, clone #167
  misc_feature 413, 475: n = A,T,C or G
ggcacgaggttcgaattttctagagaaaatgtcttgctgtggaggaaactgtggttgcggatctggctgcaagtgcggcaacggatgcggaggttgcaaaatgtacccagacttgggtttctctggtgagaccaccaccaccgagactcttgtcctcggcgttgccccggcgatggactcccagtacgaggcttccggcgagaccttcgttgccgagaatgatgcctgcaaatgcggatctgactgcaagtgcaacccttgtacctgcaaatgaacaacccataaaccctaagagtctgcaataaccctaatgttatgttaggtctggttatgtgtaataatggctgatttcgccggttgttttgccggtccttctcttcttctgctgtgtgtttttatggtttggtcataanatatcgctgcacgttttatctatgtgactatataatcaaatattattatgggtttgttttcnaaaaaaaaaaaaaaaaaaaa

SEQ ID NO: 10 - protein, 81 aa, Artificial Sequence
  protein encoded by cDNA, clone #167
MSCCGGNCGCGSGCKCGNGCGGCKMYPDLGFSGETTTTETLVLGVAPAMDSQYEASGETFVAENDACKCGSDCKCNPCTCK

SEQ ID NO: 11 - DNA, 315 bp, Artificial Sequence
  cDNA, clone #213
  misc_feature 25, 33, 160, 170, 186, 191, 242, 244, 250, 273, 302,304, 310: n = A,T,C or G
ggcacgagggcaaaagaagaatcanacaacaanaaactacaaagtttaatcaaagagaagtaagagaaacaatggccggttctaaatgtggtgactcttggagttgcgagatgaactacaacacggagtgcgacagctgcagctgtggatcagactgcanctgtgggtcnaactgcaactgttganaaatngtggtttaaaatcacatgtatgcaggaaaaactggggaaaaatatgttaananatccgngtgtgttttgaataattctcttnaccttgacttatttcctgctttgtatttntnctgttngttga

SEQ ID NO: 12 - protein, 37 aa, Artificial Sequence
  protein encoded by cDNA, clone #213
  VARIANT 30, 33: Xaa = Any Amino Acid
MAGSKCGDSWSCEMNYNTECDSCSCGSDCXCGXNCNC

SEQ ID NO: 13 - DNA, 708 bp, Artificial Sequence
  cDNA, clone #159
ggcaggaggctgaagtgatccaattgaaactttctttggttctcaagtctctttgtcctgtttttttctgagtggtgtgtgaattgtaagcttttgttaacagtaagagttttgagaaaattgtggttttgagagatggatgagactaatcatggaggttcaacaagctcactcccacctttcctcaccaaaacatatgagatggttgacgactcttcatcggactcaatcgtctcgtggagccagagcaacaagagcttcatcgtttggaatcctccagagttttccagagatcttcttccgagattcttcaagcacaacaacttctcaagctttatccgtcagcttaacacatatggttttagaaaatctgatcccgagcaatgggaatttgcgaacgatgatttcgtgagaggccaacctcatctgatgaagaacattcacagacgcaaaccagttcacagccactctttacctaatctccaagctcagcaaactccgttgacggattcggagcgacagaggatgaataaccaaatccagagacttacaaaggagaaagaaggactgctccaagagttacagaaacaagaggaggagcgtgaagggtttgagcaacaagttaaagagctaaaagatcgtttacaacacatggagaagcgtcagaagacgatggtttcgtatgtctctcaggtattggataaacca

SEQ ID NO: 14 - protein, 191 aa, Artificial Sequence
  protein encoded by cDNA, clone #159
MDETNHGGSTSSLPPFLTKTYEMVDDSSSDSIVSWSQSNKSFIVWNPPEFSRDLLPRFFKHNNFSSFIRQLNTYGFRKSDPEQWEFANDDFVRGQPHLMKNIHRRKPVHSHSLPNLQAQQTPLTDSERQRMNNQIQRLTKEKEGLLQELQKQEEEREGFEQQVKELKDRLQHMEKRQKTMVSYVSQVLDKP

SEQ ID NO: 15 - DNA, 349 bp, Artificial Sequence
  cDNA, clone #27
ggcacgagggtggtcagaccttcagaggaggcaaaactcccattagggtatcttgagacagtgaataaagacaagagcttggtcttgaaatggagtcctcagcttgaagttttatccaacaaagccagtggaccgatcaaccgatgaacgcaaagtacatacaagatgtgtggaagtgtggagttcgtgtgaagatagacaaagaaagtgggattgccaagagagaggagattgaaattagtataaaggaagtgatggaaggagagacgagcaaagggatgaaggaaaacgcaaagaaattgagagacttggctgtcaagtcactcaatgaaggatgctctacagatat

SEQ ID NO: 16 - protein, 80 aa, Artificial Sequence
  protein encoded by cDNA, clone #27
SFIQQSQWTDQPMNAKYIQDVWKCGVRVKIDKESGIAKREEIEISIKEVMEGETSKGMKENAKKLRDLAVKSLNEGCSTD

SEQ ID NO: 17 - DNA, 821 bp, Artificial Sequence
  cDNA, clone #50
gtagggttacaacggggactccgcagtagtcgctctccgatcccttcttctcccggccaaaatccgtctaaacttctcttcctcagcatcgattgcctcgtctcagctcaattctctacgttttcacgttactgcttcgtttagaaccttcacttgagtactttggtggtgggagagatgaatcacggccaacaatctggcgaggcaaagcatgaagatgacgctgcgcttacagagttccttgcttctcttatggattatactcctactattcctgatgatctagtggagcactacttggctaagagtgggtttcagtgccccgacgttcgattaataaggctagttgctgtggctacacaaaagtttgttgctgatgttgccagcgacgccccttcagcactgcaggctagaccagcacccagttgttaaagacaaaaaacagcaaaaggataagcgtttgatattgacaatggaagacctttcaaaagctttgcgtgagtacggtgtgaacgtgaagcatccagaatattttgctgatagcccttcgaccggaatggatcctgcgacaagggacgaatagaaacctgaggaagtctttgcctagaaaggatgatcatgtatgtgagatccgtgattttctatcgtgtttcagttaaaacaaacaaaactcaattctattcctagtcaccagttacgtgtatattgcttttgttgttcgtttcttgacttgcgtctctggtttcctacaacacttatctttcattcttgtaagtcttcaaatcgtgataataagataagtatccttatgagttttaaaa

SEQ ID NO: 18 - protein, 84 aa, Artificial Sequence
  protein encoded by cDNA, clone #50
MNHGQQSGEAKHEDDAALTEFLASLMDYTPTIPDDLVEHYLAKSGFQCPDVRLIRLVAVATQKFVADVASDAPSALQARPAPSC

SEQ ID NO: 19 - DNA, 742 bp, Artificial Sequence
  cDNA, clone #169
ggcacgaggtaattctcctctaatcctatcactaattgataagtacgatacaaaaaatggattcaagatttatcgacaccattccttccttgagctatattaatgacgataagagtgatgatgaatatgcgtttgtgagagctttacgtatgagtggtggtgatggagccaacagctactccgccaattctcttcttcagagaagagttttatcaatggccaaaccagtattggtaaaatacacagaagaaatgatgatgaacttagactttccaaagtacatcaaagttgctgaattgggttgttcttcgggacaaaactcatttctggctatctctgagatcatcaataccatcaatatgttgtgccaacaatcgaaccaaaacccaccagaaatcgattgttgtctgaatgatcttccgggaaacgatttcaacacgaccttcaagttcgtacctttcttccacaagaagctcatgatcacaaacagaacatcgtgtttcgtctatggagctccagggtccttctactctaggctcttctctcgcaatagcctccatttcatacactcctcttacgccctccattggctctctaaggttcctgaacaactcgagaacgatgaggaaaatgtgtacataacaagctcaagtcctcaaagtgcatacaaggcttacttgaatcaattccaaagagatttcaccatgtttctaaggttacgttcttgagaagttgtctctaa

SEQ ID NO: 20 - protein, 223 aa, Artificial Sequence
  protein encoded by cDNA, clone #169
MDSRFIDTIPSLSYINDDKSDDEYAFVRALRMSGGDGANSYSANSLLQRRVLSMAKPVLVKYTEEMMMNLDFPKYIKVAELGCSSGQNSFLAISEIINTINMLCQQSNQNPPEIDCCLNDLPGNDFNTTFKFVPFFHKKLMITNRTSCFVYGAPGSFYSRLFSRNSLHFIHSSYALHWLSKVPEQLENDEENVYITSSSPQSAYKAYLNQFQRDFTMFLRLRS

SEQ ID NO: 21 - DNA, 734 bp, Artificial Sequence
  cDNA, clone #92b
ggcacgaggaagttatccactcaaggtgggccatgctcggagccctaggctgcgtcttcccggagttgttggccaggaacggagtcaagttcggagaggcggtgtggttcaaggccggttcgcagatcttcagcgaaggagggctcgattacttgggaaacccaagcttggttcacgctcagagcattttggcgatatgggccactcaggtgatcttgatgggagctgttgaaggttacagagtcgcaggaaacgggccgttgggagaggccgaggacttgctttacccaggtggcagcttcgacccattgggcctcgctaccgacccagaggccttcgcggagttgaaggtcaaggagctcaagaacggaagattggctatgttctctatgttcggattcttcgttcaagccatcgtcaccggtaagggaccaatcgagaatcttgctgaccatttggccgatccagtcaacaacaacgcttgggccttcgccaccaatttggttcccggaaagtgagccaagtttttatctgtttgtaatttgtttttctttgcttcagtcttttgaattcgagtgagagtgaggtaagaggagaaagagtaaaaggtttgtgttggtgatgatggatggttgagactttcagatgtaaatttgtaagaccttgtatggcttatcattaatcaaataactcgtttttctcaaaaaaaaaaaaaaaaaaaaaaaaaaaaaaaa

SEQ ID NO: 22 - protein, 171 aa, Artificial Sequence
  protein encoded by cDNA, clone #92b
HEEVIHSRWAMLGALGCVFPELLARNGVKFGEAVWFKAGSQIFSEGGLDYLGNPSLVHAQSILAIWATQVILMGAVEGYRVAGNGPLGEAEDLLYPGGSFDPLGLATDPEAFAELKVKELKNGRLAMFSMFGFFVQAIVTGKGPIENLADHLADPVNNNAWAFATNLVPGK

SEQ ID NO: 23 - DNA, 486 bp, Artificial Sequence
  cDNA, clone #65b
ggcacgaggcatttgtcaaggctggcccattaaggaacactccttacgccggctccgctggctctttggccgcagctggactcgtagtcatcctcagcatgtgcctcaccatctacgggatctcttctttcaatgaaggagacccttcgatcgcaccgagtttgactttgaccggacggaagaagcagcctgaccagcttcagactgctgacggatgggctaagttcaccggagggttcttcttcggtgggatctctggcgtgacttgggcttacttccttctctacgttcttgaccttccttactacgtcaaatgaatgtagttgaaaatatatatgagtgtactttcaactctctcttgcatctttgttcttcttttgtcttgatcaagaatcttgaatcttaagggaatgattaatgtatattactatggatcttttcttaacatttaataatttatattgccttgaaaaaaaaaaaaaaaaa

SEQ ID NO: 24 - protein, 104 aa, Artificial Sequence
  protein encoded by cDNA, clone #65b
HEAFVKAGPLRNTPYAGSAGSLAAAGLVVILSMCLTIYGISSFNEGDPSIAPSLTLTGRKKQPDQLQTADGWAKFTGGFFFGGISGVTWAYFLLYVLDLPYYVK

SEQ ID NO: 25 - DNA, 303 bp, Artificial Sequence
  cDNA, clone #82
ggcacgaggcgccgacgaagctctgcaacaacaatggcgactcagatcagcaaaaagagaaagttcgttgccgatggtgttttctacgcggagctaaacgaggtcctgacaagagagctcgctgaagatggttactctggtgtcgaggtccgtgtcactcccatgcgtaccgaaatcatcatcagagccactcgtactcaaaacgttctcggtgagaagggtaggagaatcatagagttgacatcacttgtccaaaagagattcaaatttcctcaggacagtgttgagctttacgctgaaaaa

SEQ ID NO: 26 - protein, 90 aa, Artificial Sequence
  protein encoded by cDNA, clone #82
MATQISKKRKFVADGVFYAELNEVLTRELAEDGYSGVEVRVTPMRTEIIIRATRTQNVLGEKGRRIIELTSLVQKRFKFPQDSVELYAEK

SEQ ID NO: 27 - DNA, 1123 bp, Artificial Sequence
  cDNA, clone #79
ggcacgaggcctcgtgccgaattcggcacgagggaagttagtaagaaatcaaacccttgcaggcgacttggagataagaagcagattgtttaccaaatgttttctggaacaagacttgtgtaaataggtggaatcttggttggtttttgaggtattcatcaaatctaacacactcaaaagatgggatgtgtttcttcttgcttccgtgtccaagacattgatgagtacatgaatccaagtagctctgtatataggaactgtccctgcattagatgccttgctcataatttccttaacctgtatatcacggtattcaggagaggggaaacccgatctctcccgtcatcagttcaagcgactgcatcgataacttcgtcttctttccacgataactttctgtctgaagcattccgttcaactccgagacctctgccttacgatgctgatccaagatacatccgctcactcgtctcaagaagagagaaaggttctagccattcccatgaggaagctgagcctctaagaagcgatggcgacgctgcggattccgaatctttcagaggatgcagcaaatggggaaacaacaaatccgacaaagatgccaaagaagactactctagtaaatctagtctcaggatttcgaaatcaaagtcaatggttgacactgaaagcatttatgtattgtctgaagatgaagatgtgtgtcctacttgtcttgaagaatatacatcagagaatccaaagattgtaacgaaatgctgtcaccatttccatcttggttgcatttatgaatggatggagagaagtgaaaactgtcctgtctgcggaaaggtgatggagtttaacgaaacaccttgatcatcgatcattgatctgtgtcttgtatctcaactgaaaccggggaagatgaagatgacaaggcattgcaaaggagatgtttttgtaaatttggctttgttggtttgtgaatattgtcaatgacaatggtaaatatatgaagcagaaagggagaaaatatgttcctctgcttttcaacagttttacgacattggatatcttaaatatttaattacgaataataatatatcaacaagagacaagaaaaatacgtttgtttaggtaa

SEQ ID NO: 28 - protein, 225 aa, Artificial Sequence
  protein encoded by cDNA, clone #79
MGCVSSCFRVQDIDEYMNPSSSVYRNCPCIRCLAHNFLNLYITVFRRGETRSLPSSVQATASITSSSFHDNFLSEAFRSTPRPLPYDADPRYIRSLVSRREKGSSHSHEEAEPLRSDGDAADSESFRGCSKWGNNKSDKDAKEDYSSKSSLRISKSKSMVDTESIYVLSEDEDVCPTCLEEYTSENPKIVTKCCHHFHLGCIYEWMERSENCPVCGKVMEFNETP

SEQ ID NO: 29 - DNA, 964 bp, Artificial Sequence
  cDNA, clone #62
ggcacgaggctcaaatcagatcggtttccatggctgcagctgctaacaccgccgccattttcgcctctccttcgcagcctttatcctctaaaagcagttttttgtacagctcagcgattggtcaaatacaaaggagatttccaaggaggaaacttgatctgcaagtaaaagctgttgccacgactcttacaccccttgaagagaccaaagaatataagctaccttcatgggcaatgttcgagatggggacagctcctgtgtactggaaaaccatgaacggtcttcctccaaccgcaggagaaaagttgaagctattctataatccagctgcaaccaaactcactcttaacgaagactatggagttgctttcaacggaggattaaccaaccaatcatgtgtggtggggaaccaagagcaatgcttaagaaagatcgaggcaaagccgattctcccatttacactatgcagatttgcattcctaagcacgctgtgaatttgatattctcgtttaccaatggcgtggactgggacggtccatacagacttcagtttcaagtcccaagcgatggcaaacaaacctatcgagttcttcaatgaaggtctagcgaaagagttgagccaagacggagcctgcgagagagcaatatttcctgactcgaacgtagttgcgacgcggtgcacaatgatcgccaacttgacggtggaaggaggagatagatgcaatctggatttggttcctgggtgcatggatacaaattcggaacatttcaacccgtttgctaatgttgatgatggctcctgtcccctcgacttatctgattctgatgaatagagctatagcattttcttatgtaaatatatgaacccatatgttaatatcagtacgtagtatttgaatttaaatatgtatacatgtggtaacttgttgggttttactattatataagaagcttcacaatcaaa

SEQ ID NO: 30 - protein, 154 aa, Artificial Sequence
  protein encoded by cDNA, clone #62
MAAAANTAAIFASPSQPLSSKSSFLYSSAIGQIQRRFPRRKLDLQVKAVATTLTPLEETKEYKLPSWAMFEMGTAPVYWKTMNGLPPTAGEKLKLFYNPAATKLTLNEDYGVAFNGGLTNQSCVVGNQEQCLRKIEAKPILPFTLCRFAFLSTL

SEQ ID NO: 31 - DNA, 622 bp, Artificial Sequence
  cDNA, clone #215
  misc_feature 246, 514, 563, 577, 585, 609, 617, 620: n = A,T,C or G
ggcacgaggcatccaagtcccggagaaatcgatcgtagctcggtgcctttcgctttataaaatcgcatctcgacaggggaagaaaagttcgttgctttccctcaaaaatctccaatttctcgtttcatttccgttaatttatcgtttcaccgaacgcacgtcgaaaccctataacccaattggttttttgcgggtcaacttcagcttcgagttatctagggtttcgtatctgaatctgtggagaanaaaacccttcttgtgggggttacctaaattttctgaaatcagagctttaaaagggacagcttttatttgtatggaaggtctctgtcactaaactacatattgatatggaggcacaaattcatcaacttgagcaggaagcgtatactgctgttttaagggctttcaaagcgcagtcagatgctatttcttgggacaaggaaagcttgataacagagctgcgtaaagaattgagagtatctgatgacgaacatcgggagctgctgagtanggtcaaataaggacgatactatcccaaggatttacggattggagaccanggaggcggaagtcnaagttccnagacatgcagctattcagcctttntgaatgtnggntc

SEQ ID NO: 32 - protein, 57 aa, Artificial Sequence
  protein encoded by cDNA, clone #215
  VARIANT 55: Xaa = Any Amino Acid
MEAQIHQLEQEAYTAVLRAFKAQSDAISWDKESLITELRKELRVSDDEHRELLSXVK

SEQ ID NO: 33 - DNA, 626 bp, Artificial Sequence
  cDNA, clone #114
ggcacgagggtgaaatttcagctcaaatctacgactgaaaaactcattttcattgttttgtaagctacttgtttaaagcacttatcagaatggactcatgttgcaaaaaagtttcttccgactcgagctgcagcgccaagccgactacaaattgcatttgtgtccagaattccaacaaatgcccctgctgtgataacaaatcagagtgttgctgcaagcaggcgaattcctgctgcaccagtacaaataattcaagcggctgttctaaccaggctaaaacgtgttgctctaagtagatgtttgtcaactatgatttcaacattttggactgattactttcgatcttcgtttgtacgagtacaaagtaatatatgtcattctttaattcataagaattttactggatcattcaacatgcatataaattttatatgtgcttcggctatgtaaagtgaacgcagatggtgtacaataagttcatgactgctttctttactagagaggaaaaatgatgatgttttcagcatagctgctagacctacataatattgtaataaaataaaccacaaaatgttaaatatattgtaccttttaccaaaaaaaaaaaaaaaaaaaaaaaaaaaaaa

SEQ ID NO: 34 - protein, 68 aa, Artificial Sequence
  protein encoded by cDNA, clone #114
MDSCCKKVSSDSSCSAKPTTNCICVQNSNKCPCCDNKSECCCKQANSCCTSTNNSSGCSNQAKTCCSK

SEQ ID NO: 35 - protein, 1171 aa, Arabidopsis thaliana
MALQNKEEEKKKVKKLQKSYFDVLGICCTSEVPIIENILKSLDGVKEYSVIVPSRTVIVVHDSLLISPFQIAKALNEARLEANVRVNGETSFKNKWPSPFAVVSGLLLLLSFLKFVYSPLRWLAVAAVAAGIYPILAKAFASIKRPRIDINILVIITVIATLAMQDFMEAAAVVFLFTISDWLETRASYKATSVMQSLMSLAPQKAIIAETGEEVEVDEVKVDTVVAVKAGETIPIDGIVVDGNCEVDEKTLTGEAFPVPKQRDSTVWAGTINLNGYICVKTTSLAGDCVVAKMAKLVEEAQSSKTKSQRLIDKCSQYYTPAIILVSACVAIVPVIMKVHNLKHWFHLALVVLVSGCPCGLILSTPVATFCALTKAATSGLLIKSADYLDTLSKIKIVAFDKTGTITRGEFIVIDFKSLSRDINLRSLLYWVSSVESKSSHPMAATIVDYAKSVSVEPRPEEVEDYQNFPGEGIYGKIDGNDIFIGNKKIASRAGCSTVPEIEVDTKGGKTVGYVYVGERLAGFFNLSDACRSGVSQAMAELKSLGIKTAMLTGDNQAAAMHAQEQLGNVLDVVHGDLLPEDKSRIIQEFKKEGPTAMVGDGVNDAPALATADIGISMGISGSALATQTGNIILMSNDIRRIPQAVKLARRARRKVVENVCLSIILKAGILALAFAGHPLIWAAVLVDVGTCLLVIFNSMLLLREKKKIGNKKCYRASTSKLNGRKLEGDDDYVVDLEAGLLTKSGNGQCKSSCCGDKKNQENVVMMKPSSKTSSDHSHPGCCGDKKEEKVKPLVKDGCCSEKTRKSEGDMVSLSSCKKSSHVKHDLKMKGGSGCCASKNEKGKEVVAKSCCEKPKQQVESVGDCKSGHCEKKQAEDIVVPVQIIGHALTHVEIELQTKETCKTSCCDSKEKVKETGLLLSSENTPYLEKGVLIKDEGNCKSGSENMGTVKQSCHEKGCSDEKQTGEITLASEEETDDQDCSSGCCVNEGTMKQSFDEKKHSVLVEKEGLDMETGFCCDAKLVCCGNTEGEVKEQCRLEIKKEEFCKSGCCGEEIQTGEITLVSEEETESTNCSTGCCVDKEEVTQTCHEKPASLVVSGLEVKKDEHCESSHRAVKVETCCKVKIPEACASKCRDRAKRHSGKSCCRSYAKELCSHRHHHHHHHHHHHVSA

SEQ ID NO: 36 - protein, 951 aa, Arabidopsis thaliana
MASKKMTKSYFDVLGICCTSEVPLIENILNSMDGVKEFSVIVPSRTVIVVHDTLILSQFQIVKALNQAQLEANVRVTGETNFKNKWPSPFAVVSGILLLLSFFKYLYSPFRWLAVAAVVAGIYPILAKAVASLARFRIDINILVVVTVGATIGMQDYTEAAVVVFLFTIAEWLQSRASYKASAVMQSLMSLAPQKAVIAETGEEVEVDELKTNTVIAVKAGETIPIDGVVVDGNCEVDEKTLTGEAFPVPKLKDSTVWAGTINLNGYITVNTTALAEDCVVAKMAKLVEEAQNSKTETQRFIDKCSKYYTPAIILISICFVATPFALKVHNLKHWVHLALVVLVSACPCGLILSTPVATFCALTKAATSGLLIKGADYLETLAKIKIVAFDKTGTITRGEFIVMDFQSLSEDISLQSLLYWVSSTESKSSHPMAAAVVDYARSVSVEPKPEAVEDYQNFPGEGIYGKIDGKEVYIGNKRIASRAGCLSVPDIDVDTKGGKTIGYVYVGETLAGVFNLSDACRSGVAQAMKELKSLGIKIAMLTGDNHAAAMHAQEQLGNAMDIVRAELLPEDKSEIIKQLKREEGPTAMVGDGLNDAPALATADIGISMGVSGSALATETGNIILMSNDIRRIPQAIKLAKRAKRKVVENVVISITMKGAILALAFAGHPLIWAAVLADVGTCLLVILNSMLLLSDKHKTGNKCYRESSSSSVLIAEKLEGDAAGDMEAGLLPKISDKHCKPGCCGTKTQEKAMKPAKASSDHSHSGCCETKQKDNVTVVKKSCCAEPVDLGHGHDSGCCGDKSQQPHQHEVQVQQSCHNKPSGLDSGCCGGKSQQPHQHELQQSCHDKPSGLDIGTGPKHEGSSTLVNLEGDAKEELKVLVNGFCSSPADLAITSLKVKSDSHCKSNCSSRERCHHGSNCCRSYAKESCSHDHHHTRAHGVGTLKEIVIE

SEQ ID NO: 37 - protein, 760 aa, Arabidopsis thaliana
MAEGEESKKMNLQTSYFDVVGICCSSEVSIVGNVLRQVDGVKEFSVIVPSRTVIVVHDTFLISPLQIVKALNQARLEASVRPYGETSLKSQWPSPFAIVSGVLLVLSFFKYFYSPLEWLAIVAVVAGVFPILAKAVASVTRFRLDINALTLIAVIATLCMQDFTEAATIVFLFSVADWLESSAAHKASIVMSSLMSLAPRKAVIADTGLEVDVDEVGINTVVSVKAGESIPIDGVVVDGSCDVDEKTLTGESFPVSKQRESTVMAATINLNGYIKVKTTALARDCVVAKMTKLVEEAQKSQTKTQRFIDKCSRYYTPAVVVSAACFAVIPVLLKVQDLSHWFHLALVVLVSGCPCGLILSTPVATFCALTKAATSGFLIKTGDCLETLAKIKIVAFDKTGTITKAEFMVSDFRSLSPSINLHKLLYWVSSIECKSSHPMAAALIDYARSVSVEPKPDIVENFQNFPGEGVYGRIDGQDIYIGNKRIAQRAGCLTDNVPDIEATMKRGKTIGYIYMGAKLTGSFNLLDGCRYGVAQALKELKSLGIQTAMLTGDNQDAAMSTQEQLENALDIVHSELLPQDKARIIDDFKIQGPTMMVGDGLNDAPALAKADIGISMGISGSALATETGDIILMSNDIRKIPKGMRLAKRSHKKVIENVVLSVSIKGAIMVLGFVGYPLVWAAVLADAGTCLLVILNSMMLLRDEREAVSTCYRASTSSPVKLEEDEVEDLEVGLLQKSEETSKKSCCSGCCSGPKDNQQK

SEQ ID NO: 38 - protein, 819 aa, Arabidopsis thaliana
MEPATLTRSSSLTRFPYRRGLSTLRLARVNSFSILPPKTLLRQKPLRISASLNLPPRSIRLRAVEDHHHDHHHDDEQDHHNHHHHHHQHGCCSVELKAESKPQKMLFGFAKAIGWVRLANYLREHLHLCCSAAAMFLAAAVCPYLAPEPYIKSLQNAFMIVGFPLVGVSASLDALMDIAGGKVNIHVLMALAAFASVFMGNALEGGLLLAMFNLAHIAEEFFTSRSMVDVKELKESNPDSALLIEVHNGNVPNISDLSYKSVPVHSVEVGSYVLVGTGEIVPVDCEVYQGSATITIEHLTGEVKPDEAKAGDRVPGGARNLDGRMIVKATKAWNDSTLNKIVQLTEEAHSNKPKLQRWLDEFGENYSKVVVVLSLAIAFLGPFLFKWPFLSTAACRGSVYRALGLMVAASPCALAVAPLAYATAISSCARKGILLKGAQVLDALASCHTIAFDKTGTLTTGGLTCKAIEPIYGHQGGTNSSVITCCIPNCEKEALAVAAAMEKGTTHPIGRAVVDHSVGKDLPSIFVESFEYFPGRGLTATVNGVKTVAEESRLRKASLGSIEFITSLFKSEDESKQIKDAVNASSYGKDFVHAALSVDQKVTLIHLEDQPRPGVSGVIAELKSWARLRVMMLTGDHDSSAWRVANAVGITEVYCNLKPEDKLNHVKNIAREAGGGLIMVGEGINDAPALAAATVGIVLAQRASATAIAVADILLLRDNITGVPFCVAKSRQTTSLVKQNVALALTSIFLAALPSVLGFVPLWLTVLLHEGGTLLVCLNSVRGLNDPSWSWKQDIVHLINKLRSQEPTSSSSNSLSSAH

(19) United States
(12) Patent Application Publication
(10) Pub. No.: US 2007/0050868 A1
(43) Pub. Date: Mar. 1, 2007

(54) Agents for phytoremediation

(76) Inventors: Nathalie Verbruggen, Brussels (BE); Catherine Bernard, Brussels (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/547,949

(22) PCT Filed: Mar. 8, 2004
(86) PCT No.: PCT/BE04/00035

§ 371 Date: Sep. 25, 2006

Related U.S. Application Data
(60) Provisional application No. 60/453,271, filed on Mar. 7, 2003.

(51) Int. Cl. A01H 1/00; C07H 21/04; C12N 15/82; C12N 5/04; C07K 14/415

(52) U.S. Cl. 800/288; 75/721; 435/419; 435/468; 530/370; 536/23.6

(57) ABSTRACT

The present invention is related to new isolated and purified polynucleotide and polypeptide sequences of Thlaspi caerulescens, as well as their potential application in phytoremediation.

### FIELD OF THE INVENTION

The present invention is related to nucleic acids involved in heavy metal metabolism and able to be used for improving phytoremediation of contaminated media.

The present invention also concerns nucleic acids which may possibly be used in phytoextraction.

### STATE OF THE ART

Specific heavy metals such as Zn, Cu, Mn, Fe, . . . are essential oligoelements necessary for living organisms at low concentrations but they are toxic for them at elevated concentrations. Other heavy metals, such as Cd, Hg, Pb, . . . are not micronutrients and are toxic for living organisms even at much lower concentrations.

While some heavy metals are naturally present in soils, abnormal accumulation of heavy metals at high concentrations often result from human activities such as mining, intensive agriculture or energy production.

Contamination of soil and water with toxic concentrations of heavy metals is a world-wide problem, which can be detrimental to human health and ecosystem functions. The major entry of heavy metals like cadmium in human is, for non-smokers, through the food chain, by eating contaminated plants.

The most common way to solve this problem of contamination is to remove and bury contaminated soil or to isolate the contaminated area. However, this solution is of great cost and alternatives are under investigation.

Among them, phytoremediation and phytoextraction, which use plants able to grow in soil containing high concentrations of heavy metals, represent potentially cost-effective and environmentally friendly ways of decontaminating sites (Salt et al., Annu. revendication. plant Physiol. Plant Mol. Biol, 49, P463-468, 1998).

Plants to be used for decontamination should be tolerant and should accumulate heavy metals in their harvestable part. For efficient phytoextraction, these plants should also present a high biomass.

Extensive variation exists among plants in the ability to tolerate elevated concentrations of heavy metals in the soil, but the physiological mechanisms and genetic control of these differences are still poorly understood.

Two strategies of adaptation have evolved, namely exclusion and hyperaccumulation (salt et al. 1998). In the hyperaccumulation strategy, heavy metals are also accumulated in the aerial parts. Hyperaccumulator species have been identified for many heavy metals and are defined as plants containing in their aerial parts at least 100 times more metals than other plants grown on contaminated soil.

Concerning more particularly cadmium, only two hyperaccumulator species have been described until now: Thlaspi caerulescens and Arabidopsis halleri. For Cd, hyperaccumulation is defined as an accumulation in the aerial part which is above 0.01% of the dry weight.

The metal tolerance and hyperaccumulation capacity of some plant species is so high that it has been proposed to directly exploit them for phytoremediation, i.e. the removal of metals from contaminated soil or aqueous media (Salt et al., 1998).

However, practical difficulties have still to be solved in order to efficiently use said hyperaccumulators, among which are the slow growth rate and low growth habit (rosette) of many hyperaccumulators, and the specific nature of their metal tolerance (Ernst 1995).

### AIMS OF THE INVENTION

The present invention aims to provide polynucleotide and polypeptide sequences associated to cadmium tolerance and accumulation in plant cells.

The present invention also aims to provide polynucleotide sequences and regulatory sequences containing said polynucleotide sequences able to improve cadmium tolerance of plant cells, when expressed in foreigner organisms.

The present invention aims to provide a recombinant plant expressing said polynucleotidic sequences which could be used for phytoremediation applications and/or for phytoextraction applications.

A last object of the present invention is to provide such a plant or plant cell or tissue expressing said polynucleotide sequence which presents a sufficient growth rate for phytoremediation applications and from which cadmium can be easily extracted for recycling purposes.

### DEFINITIONS

It is meant by “phytoremediation” the use of green plants to remove pollutants from the environment or to render them harmless. Phytoextraction, phytodegradation, rhizofiltration, phytostabilisation, phytovolatilisation and the use of plants to remove pollutants from air (Salt et al., 1998).

Phytoextraction is the use of pollutant-accumulating plants to remove metals or organics from soil by concentrating them in the harvestable parts.

Preferably, said phytoremediation is a hyperaccumulation, which means the capacity of said plants to accumulate heavy metals in greater quantities than a plant normally does. It is meant by “hyperaccumulator” a plant containing in their aerial parts at least 10 times, preferably at least 100 times more metals than other plants grown on contaminated soil (for cadmium the threshold is 100 μg/g dry weight (0.01%) (Ref. Brooks et al. Trends in Plant Science, vol. 3 no. 9 p. 359-362)).

The term <<polypeptide >> refers to any peptide or protein comprising two or more amino acids joined to each other by peptide bonds or modified peptide bonds, i.e., peptide isosteres. This term “polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslational natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-linkings, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino of amino acids to proteins such as arginylation, and ubiquitination. See, for instance, PROTEINS—STRUCTURE AND MOLECULAR PROPERTIES, 2^{nd} Ed., T. E. Creighton, W. H. Freeman and Company, New York, 1993 and Wolt, F., Posttranslational Protein Modifications: Perspectives and Prospects, pp. 1-12 in POSTTRANSLATIONAL COVALENT MODIFICATION OF PROTEINS, B. C. Johnson, Ed., Academic Press, New York, 1983; Seifter et al., “Analysis for protein modifications and non-protein cofactors”, Meth. Enzymol. (1990) 182 : 626-646 and Rattan et al., “Protein Synthesis: Posttranslational Modifications and Aging”, Ann NY Acad Sci (1992) 663 : 48-62.

The term “polynucleotide” generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. The term “Polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications have been made to DNA and RNA; thus, “Polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

The term “variant” as used herein, refers to a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more substitutions (preferably conservative), additions and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants should retain one or more of the biological activities of the reference polypeptide. For instance, they should have similar antigenic or immunogenic activities as the reference polypeptide. Antigenicity can be tested using standard immunoblot experiments, preferably using polyclonal sera against the reference polypeptide. The immunogenicity can be tested by measuring antibody responses (using polyclonal sera generated against the variant polypeptide) against purified reference polypeptide in a standard ELISA test. Preferably, a variant would retain all of the above biological activities.

The term “identity” is a measure of the identity of nucleotide sequences or amino acid sequences. In general, the sequences are aligned so that the highest order match is obtained. “Identify” per se has an art-recognised meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds, Humana Press, New Jersey, 1994; SEQUENCE ANALYSIS IN MOLECULAR BIOLOGY, von Heijne, G., Academic Press, 1987; and SEQUENCE ANALYSIS PRIMER, Gribskov, M. and Devereux, J., eds, M Stockton Press, New York, 1991). While there exist a number of methods to measure identity between two polynucleotide or polypeptide sequences, the term “identity” is well known to skilled artisans (Carillo, H., and Lipton, D., SIAM J Applied Math (1.998) 48 : 1073). Methods commonly employed to determine identity or similarity between two sequences include, but are not limited to those disclosed in Guide to Huge Computers, Martin J. Bishop, ed., Academic Press, San Diego, 1994, and Carillo, H., and Lipton, D., SIAM J Applied Math (1988) 48 : 1073. Methods to determine identity and similarity are codified in computer programs. Preferred computer program methods to determine identity and similarity between two sequences include, but are not limited to, GCG program package (Devereux, J., et al., J Molec Biol (1990) 215 : 403). Most preferably, the program used to determine identity levels was the GAP program, as was used in the Examples hereafter.

As an illustration, by a polynucleotide having a nucleotide sequence having at least, for example, 95% “identity” to a reference nucleotide sequence is intended that the nucleotide sequence of the polynucleotide is identical to the reference sequence except that the polynucleotide sequence may include an average up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

Fragments of polypeptides are also included in the present invention. A fragment is a polypeptide having an amino acid sequence that is the same as a part, but not all, of the amino acid sequence of the aforementioned polypeptides. As with polypeptides, fragment may be “free-standing” or comprised within a larger polypeptide of which they form a part or region, most preferably as a single continuous region. Representative examples of polypeptide fragments of the invention, include, for example, fragments from about amino acid number 752 to about amino acid number 1030 of the polypeptide. In this context “about” includes the particularly recited ranges larger or smaller by several, 5, 4, 3, 2 or 1 amino acid at either extreme or at both extremes.

Preferred fragments include, for example, truncated polypeptides having the amino acid sequence of polypeptides, except for deletion of a continuous series of residues that includes the amino terminus, or a continuous series of residues that includes the carboxyl terminus and/or transmembrane region or deletion of two continuous series of residues, one including the amino terminus and one including the carboxyl terminus. Also preferred are fragments characterised by structural or functional attributes such as fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments. Biologically active fragments are those that mediate the protein activity, including those with a similar activity or an improved activity, or with a decreased undesirable activity. Also included are those that are antigenic or immunogenic in an animal or in a human.

### SUMMARY OF THE INVENTION

The present invention is related to isolated and purified genetic sequences from Thlaspi caerulescens, said sequence being selected from the group consisting of SEQ.ID.NO.1 to SEQ.ID.NO.32 as well as other sequences isolated from unknown (micro)-organisms SEQ.ID.NO.33 and SEQ.ID.NO.34.

The present invention is also related to genetic sequences which present an homology higher than 80%, 85%, 90%, 95%, with SEQ.ID.NO.1 or SEQ.ID.NO.5 or SEQ.ID.NO.7 or their complementary strand.

The present invention is also related to genetic sequences which present an homology higher than 75%, 80%, 85%, 90%, 95%, with SEQ.ID.NO.3 or SEQ.ID.NO.33 or their complementary strand.

The present invention is also related to genetic sequences which present an homology higher than 95% with SEQ.ID.NO.9 or SEQ.ID.NO.13 or their complementary strand.

The present invention is also related to genetic sequences which present an homology higher than 85%, 90%, 95%, with SEQ.ID.NO.11 or SEQ.ID.NO.15 or their complementary strand.

The present invention is also related to genetic sequences which present an homology higher than 95% with SEQ.ID.NO.17 or their complementary strand.

The present invention is also related to genetic sequences which present an homology higher than 75%, 80%, 85%, 90%, 95%, with SEQ.ID.NO.19 or SEQ.ID.NO.27 or their complementary strand.

The present invention is also related to genetic sequences which present an homology higher than 80%, 85%, 90%, 95%, with SEQ.ID.NO.29 or their complementary strand.

The present invention is also related to genetic sequences which present an homology higher than 95% with SEQ.ID.NO.31 or their complementary strand.

The present invention is also related to genetic sequences which present an homology higher than 98% with SEQ.ID.NO.23 or higher than 99% with SEQ.ID.NO.21 or SEQ.ID.NO.25 or their complementary strand.

The present invention is also related to polypeptide sequences encoded by the polynucleotide sequences mentioned hereabove, their active fragments and variants.

Active fragments or variants of the polypeptide sequences according to the invention are molecules which present the same activity with one or more genetic modifications (such as deletion or addition of one or more amino-acids) in the complete sequences mentioned hereabove, such as naturally occurring allelic variants. Such modifications do not modify the above mentioned percentage of homology or sequence identity.

An example of said fragments is the portion of SEQ ID No 4 starting from aminoacid 719 up to aminoacid 1134 which comprises the COOH terminal portion of sequence SEQ ID No 4; Said terminal portion comprising amino acids that are able to bind heavy metals.

Said variants are also molecules which present a similar activity to the polypeptides according to the invention through the same biochemical pathway and acting similarly upon the same active site.

The polypeptides can be also integrated as “native” protein or are part of a fusion protein or may advantageously include additional amino-acid sequences which contain secretory or leader sequences, prosequences, sequences which elute in purification such as multiple histidinoresidue or an additional sequence for stability during recombinant production (tag His in the C-terminal sequence).

Said polypeptides may comprise also marker sequences which facilitate purification of the fused polypeptide with a sequence as an hexa-histidine peptide as provided in the PQE vector (Invitrogen Inc.) and described by Gentz et al., Proceeding National Academic of Science of the USA, 1989, Vol. 86, pp. 821-824) or an HA tag or glutathione-S transferase. The corresponding polynucleotide may also contain non-coding 5′ and 3′ sequences such as transcribed non-translated sequences, splicing and poly-adenylation signal and ribosome binding sites.

Another aspect of the present invention is related to a vector comprising the polynucleotide or polypeptide according to the invention, said vector being preferably a plasmid, a virus, a liposome or a cationic vesicle able to transfect a cell and to obtain the expression of said polynucleotide by said cell.

The vector according to the invention may be a shuttle vector for suitable transformation of different types of cells.

A further aspect of the present invention concerns the cell (prokaryotic or eukaryotic cell) or the plant transformed by or comprising the vector according to the present invention and their use for phytoremediation (including phytoextration) of media (such as soils), contamined by heavy metals.

### DETAILED DESCRIPTION OF THE INVENTION

Material and methods

Plant cDNA Bank

A cDNA bank from leaves of one of the best Cd hyperaccumulator population of Thlaspi caerulescens (Roosens et al. Plant cell and Envir. Vol 26, p 1657-1672) was integrated in the pYX212 vector. The pYX212 vector is a yeast/E. coli shuttle vector for expression in S. cerevisiae sold by R&D ingenius company (Madison, USA). Insert was under the activity of the triose phosphate isomerase promoter, which is one of the strongest constitutive promoter in yeast. pYX212 is a 2μ plasmid, replicates autonomously in yeast, being maintained at 25-100 copies per cell. The plant cDNA were cloned between the EcoRI and the XhoI sites. The selection marker was URA3 in yeast.

Yeast Strain Used for Transformation

The Saccharomyces cerevisiae wild-type strain used for transformation experiments was BY4741 ATCC Number 201388 (“Yeast Genetic Stock Collection” in the ATCC Global Bioresource Center).

E. coli Strain Used for Transformation

The E. coli strain used for experiments was DH5 alpha ATCC Number 53868.

Plasmid Isolation from Yeast and Transformation of E. coli Strain

Small scale isolation of plasmid DNA from yeast for transformation in E. coli was done according to the method disclosed in Current Protocols in Molecular Biology 1993 John Wiley & Sons; Inc (Chapter 13).

Transformation of E. coli was done by electroporation according to the method described in Current Protocols in Molecular Biology 1993 John Wiley & Sons, Inc (Chapter 1).

Plasmid Isolation from E. coli and Retransformation of Yeast

Plasmid isolation from E. coli was performed with the Wizard Plus Miniprep DNA Purification Systems (Promega).

Transformation of yeast by Li Cl. Gietz, R. D & Schiestl, R. H. (1995) has been carried out using the technique disclosed in Methods Mol. Cell. Biol. 5, 255-269.

Results

The plant cDNA library of Thlaspi caerulescens was screened in the Saccharomyces cerevisiae wild-type yeast strain BY4741. The transformants were plated on minimal medium supplemented with cadmium. From 430.000 S. cerevisiae transformants, 200 clones growing on 15 μM cadmium were identified. To confirm the correlation between the cadmium tolerance phenotype and the expression of the plant cDNA, plasmids have been rescued and yeast has been re-transformed. From 200 plasmids, 150 have been re-tested and 110 have been reconfirmed by drop tests on 20 μM cadmium and further sequenced. From sequence analysis, 19 different non-redundant cDNAs were identified encoding proteins displaying significant homology with:
group I: metal detoxification related proteins:
phytochelatin synthase 1;
2 different isoforms of metallothionein type 3 (type 3a and type 3b);
metallothionein type 2;
metallothionein type 1;
metallothionein related protein;
group II: metal transport related proteins corresponding to Cd/Zn transporting P-type ATPases;
group III: sigballing pathway related proteins:
a heat shock transcription factor;
transcription factor IID;
group IV: other proteins:
SAM: salicylic acid carboxylmethyl transferase;
chlorophyll a/b binding proteins;
a 40S ribosomal protein;
Photosystem I subunit.

3 proteins were classified in a last group V with unknown function.

The results of sequence analysis and functional classification of said identified cDNAs are presented in Table 1.

It should be noted that cDNAs of group II correspond to four truncated cDNAs encoding proteins with similarity to the C-terminal region of utative heavy-metal P-type ATPases, also called in the present description “CPx-ATPases”.

Said results show that the majority of the identified cDNAs encode proteins known to have a potential role in heavy metal tolerance as metal binding proteins, metallothioneins and phytochelatins, and heavy metal binding domain of putative CPx-ATPases that display Zn²⁺/Co²⁺/Cd²⁺/Pb²⁺ substrate specificity.

Analysis of cDNAs Encoding Truncated Putative CPx-ATPases

In Silico Analysis:

In silico analysis of the previously identified cDNAs encoding truncated putative CPx-ATPases showed a higher similarity with the C-terminus of A. thaliana HMA4 and these corresponding sequences in T. caerulescens were therefore hereafter called “TcHMA4”.

The deduced TcHMA4 proteins encoded by cDNAs 71, 165 and 199 lacked the putative catalytic domain while keeping the putative heavy metal binding domains. In contrast, cDNA 64, the longest isolated, encoded a protein which contains the ATP-binding site.

Heterologous Expression in Yeast:

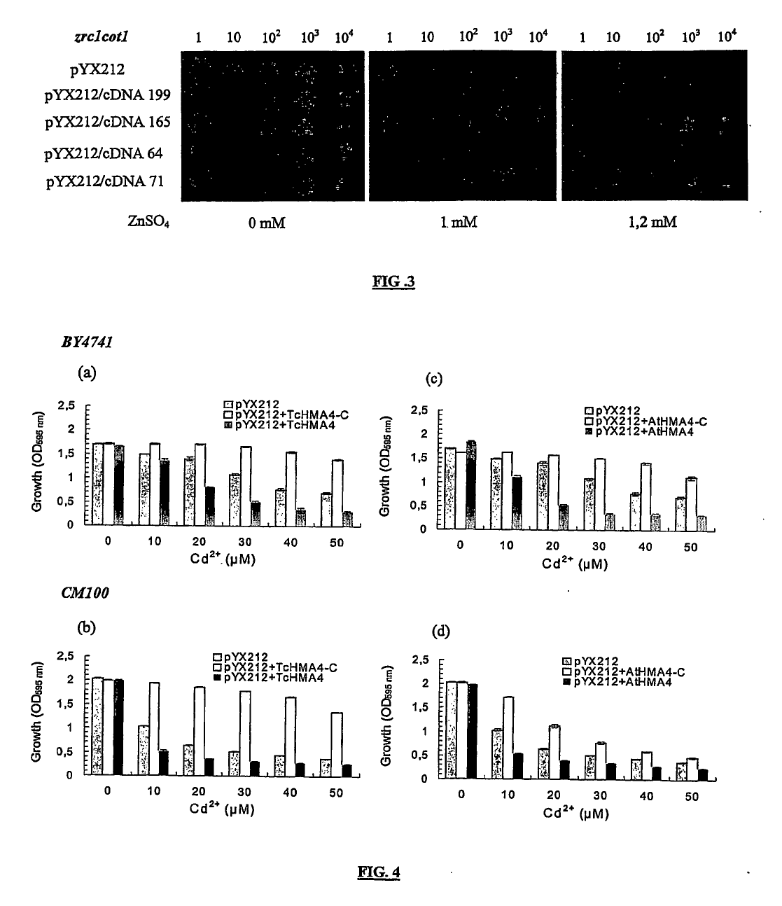

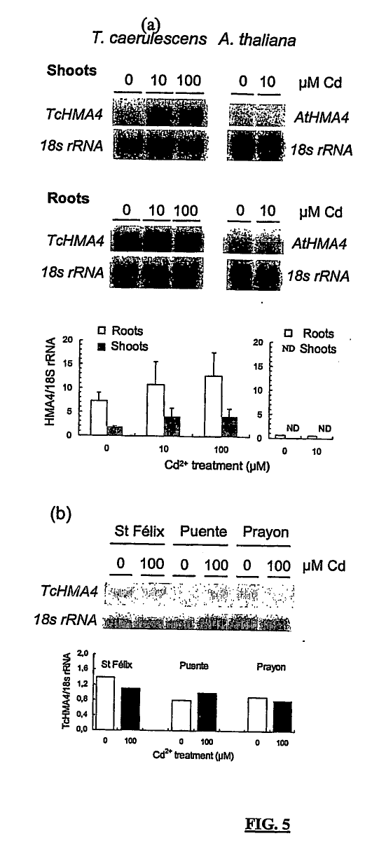

To confirm and to compare the ability of Thlaspi cDNAs 64, 71, 165 and 199 to increase cadmium tolerance to S. cerevisiae, BY4741 cells expressing these cDNAs were further analysed for their cadmium tolerance (FIG. 2: Evaluation of growth in the presence of cadmium. Transformants of the yeast strain BY4741 containing empty plasmid pYX212 as negative control and pYX212 with Thlapsi cDNAs 199, 165, 64 and 71 were grown in liquid minimal medium overnight. Cultures were adjusted to A₆₀₀ of 1 and serially 10-fold diluted in water. 5 μl aliquots of each dilution were spotted either on non-selective cadmium plates or on plates with 20 and 40 μM CdSO₄. After three days of incubation at 30° C., plates were photographed. Dilutions are indicated at the top of the figures).

Control cells (carrying the expression vector pYX212) grew normally in the absence of cadmium but were highly sensitive to cadmium and no growth was observed on 40 μM CdSO₄.

Cells expressing cDNAs 71, 165 and 199 were able to grow on 20 and 40 μM CdSO₄.

Expression of cDNAs 71 and 165 afforded the best cadmium tolerance. Growth was still observed at dilution 10³ (˜125 cells/5 μl aliquot) on 40 μM CdSO₄.

In contrast, cells expressing cDNA 64 were more sensitive compared to cells expressing the three other cDNAs and no growth was observed on 40 μM CdSO₄.

Because growth tests with the wild type strain BY4741 require a high zinc concentration (11 mM ZnSO₄), zinc related phenotype was also tested in the zinc hypersensitive zrclcot1 double mutant. This yeast strain lacks two vacuolar transporters (ZNT1 and COT1, which confer Zn resistance by its sequestration into the vacuole (Li and Kaplan, 1998)) and was more sensitive to zinc than the parental wild type strain (MacDiarmid et al., 2003).

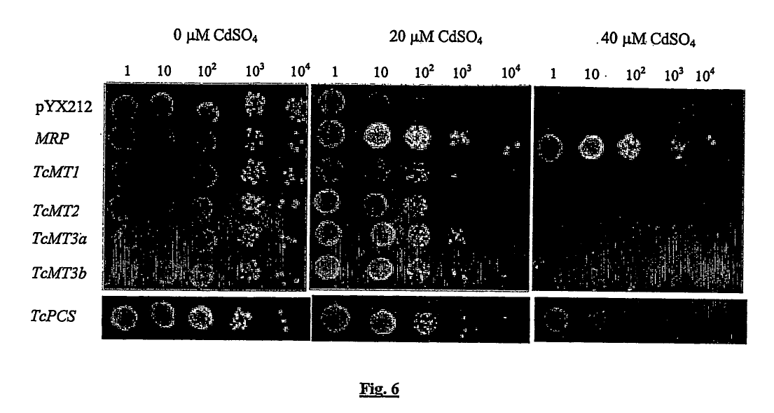

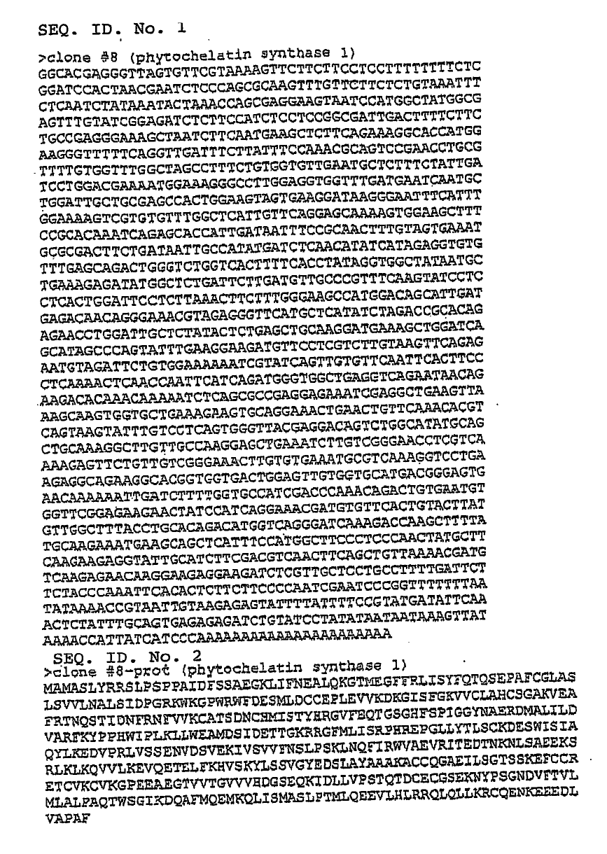

The profile of growth of transformed zrclcot1 on Zn was similar to the one of transformed BY4741 on Cd. Yeast cells expressing cDNAs 71 and 165 showed the best zinc tolerance. No difference in growth was observed between control cells and cells expressing cDNA 64 at the used concentrations (FIG. 3: Evaluation of growth in the presence of zinc. Transformants of the zinc hypersensitive zrclcot1 double mutant (parental strain BY4741) containing control plasmid pYX212 and pYX212 with Thlapsi cDNAs 199, 165, 64 and 71 were grown in liquid minimal medium overnight. Cultures were adjusted to A₆₀₀ of 1 and serially 10-fold diluted in water. 5 μl aliquots of each dilution were spotted either on non-selective zinc plates or on plates with 1 and 1.2 mM ZnSO₄. After three days of incubation at 30° C., plates were photographed. Dilutions are indicated at the top of the figures).

Cloning of a TcHMA4 Full-Length Coding Sequence

To isolate a full-length cDNA, a RT-PCR approach was used. As the cDNA 71 (with the cDNA 165) confers the best tolerance to cadmium and zinc when overexpressed in yeast, this cDNA was completely sequenced and used as a starting sequence to determine reverse primers.

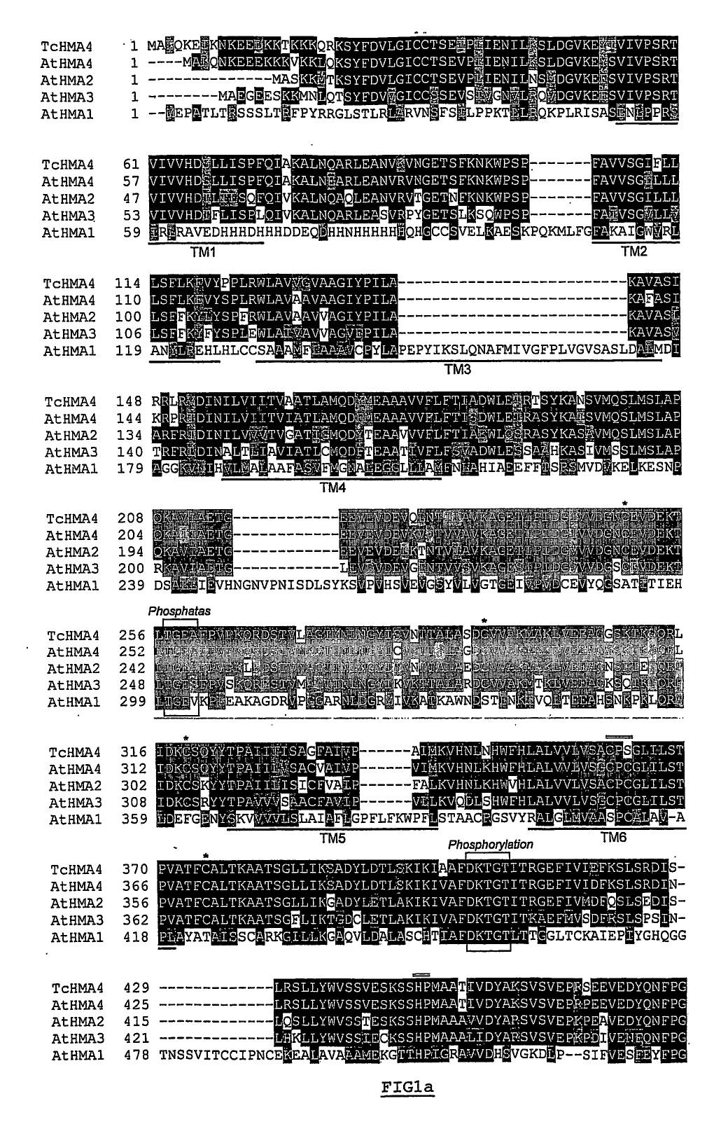

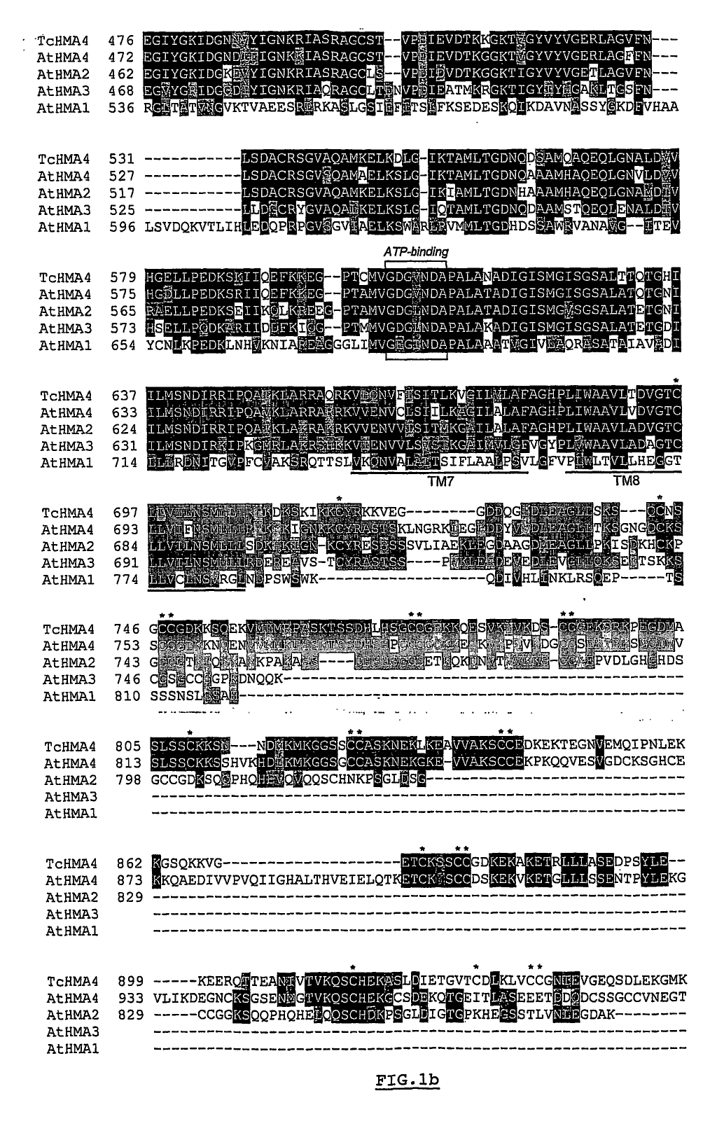

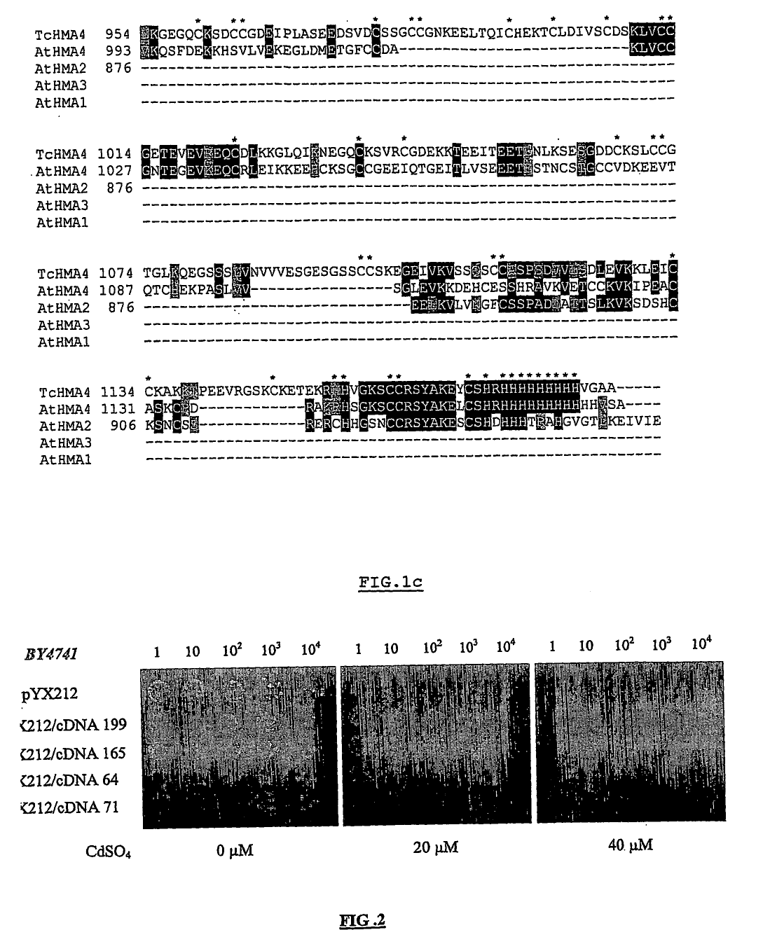

Since the highest homology was found with the A. thaliana HMA4, the T. caerulescens corresponding gene was named TcRM4 (SEQ ID NO. 4 (FIG. 1)).

Sequence Analysis of TcMA4:

The amino-acid sequence deduced from TcHMA4 aligned well with several A. thaliana HMAs. The TcHMA4 deduced amino acid sequence displayed 69% identity and 76% similarity with the AtHMA4 sequence.

The TcHMA4 and AtHMA4 deduced protein sequences display the characteristic features of CPx-ATPases in addition of the conserved motifs of P-type ATPases (the DKTGT phosphorylation motif and the GDGxNDx ATP binding motif).

Transmembrane (TM) predictions were used from various programs together with the hydropathy calculated by the Kyte-Doolittle algorithm (Kyte and Doolittle, 1982), as well as with the information from the location of conserved sequences to predict the locations of transmembrane domains in HMA4.

TcHMA4 as AtHM4 are predicted to contain eight transmembrane domains with a small cytoplasmic loop between TM domain 4 and 5 and a large cytoplasmic loop between TM domains 6 and 7, which are characteristics of CPx-ATPases.

The CPx motif (C361PS in TcHMA4; C₃₅₇PC in AtHMA4) was found in the sixth transmembrane domain as well as a specific HP(H₄₄₅ in TcHMA4; H₄₄₁ in AtHMA4) sequence located in the large predicted cytoplasmic domain, 39 amino acids downstream of the phosphorylation site.

Besides features typical of CPx-ATPases, the TcHMA4 sequence also displayed significant differences from those, which it shared with AtHMA4. Both TcHMA4 and AtHMA4 lacked the N-terminal metal associated domain (GMTCxxC).

Nevertheless, both the pfam and PROSITE databases recognise a “heavy metal associated domain” in the N-termini of Tc- and At-HMA4.

The presence of a long COOH extension after the eight transmembrane domain was another particular feature that TcHMA4 shared with AtHMA4 (478 amino acids for TcHMA4 and 470 amino acids for AtHMA4) and to a lesser extent with AtHMA2 (267 amino acids). All these three peptides also contained three additional cysteine motifs—C(x)₄C, C(X)₃₋₅CC, CC—and a His rich domain within their extended C-terminus which could be involved in heavy metal binding.

The His rich domain was present in AtHMA1, where it was associated with a single CC dipeptide, but in this case in the N-terminal domain. The TcHMA4 C-terminal fragment corresponding to the cDNA identified during the screening in yeast, consisted of TcHMA4 residues 758 to 1186 and hence lacked the putative catalytic domains while keeping the putative heavy metal binding domains. These could be responsible for the higher tolerance to Cd²⁺ conferred to yeast that overexpressed that peptide.

Metal Tolerance in Yeast Expressing Truncated and Full Length HMA4 Coding Sequences

Cadmium Tolerance Test:

To investigate cadmium specificity of HMA4, heterologous expression in S. cerevisiae was carried out. The wild type strain BY4741 was transformed with the pYX212 vector expressing TcHMA4-C and TcRM4 coding sequences under the control of the strong constitutive TPI (triose phosphate isomerase) promoter. Growth was monitored on solid and in liquid media containing various cadmium concentrations.

Expression of TcHMA4-C allowed S. cerevisiae cells to grow in the presence of 15 μM on solid up to 50 μM CdSO₄ on liquid media, which reduced growth of control cells bearing the pYX212 cloning vector.

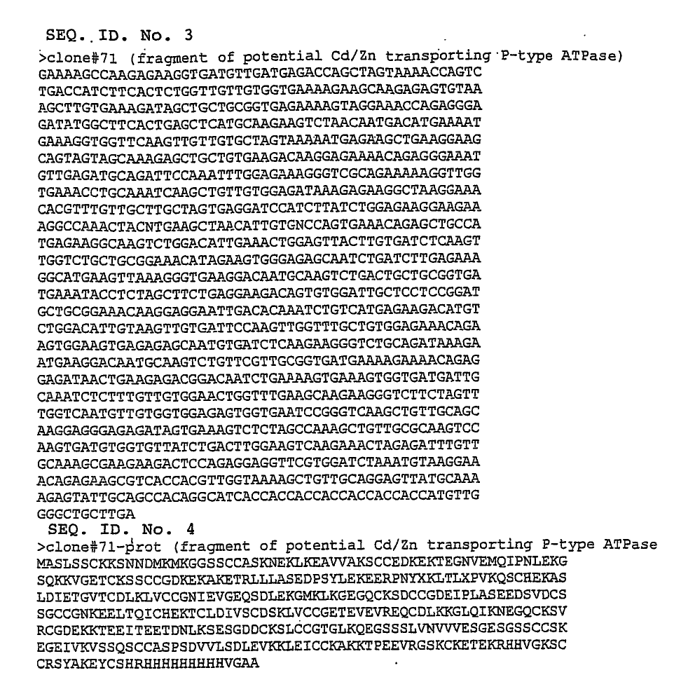

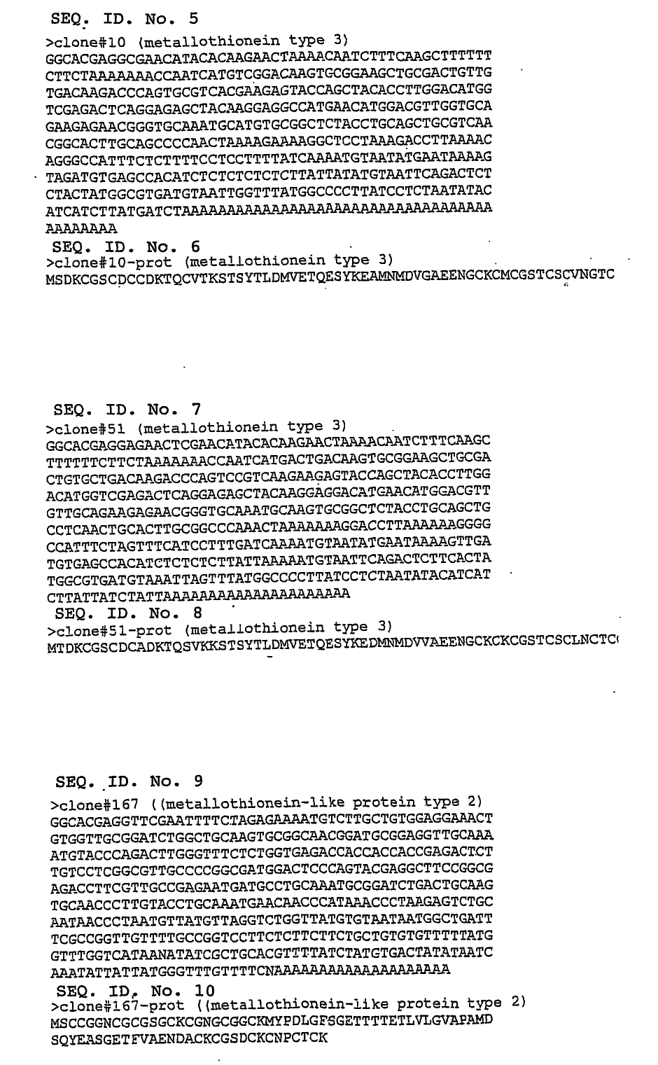

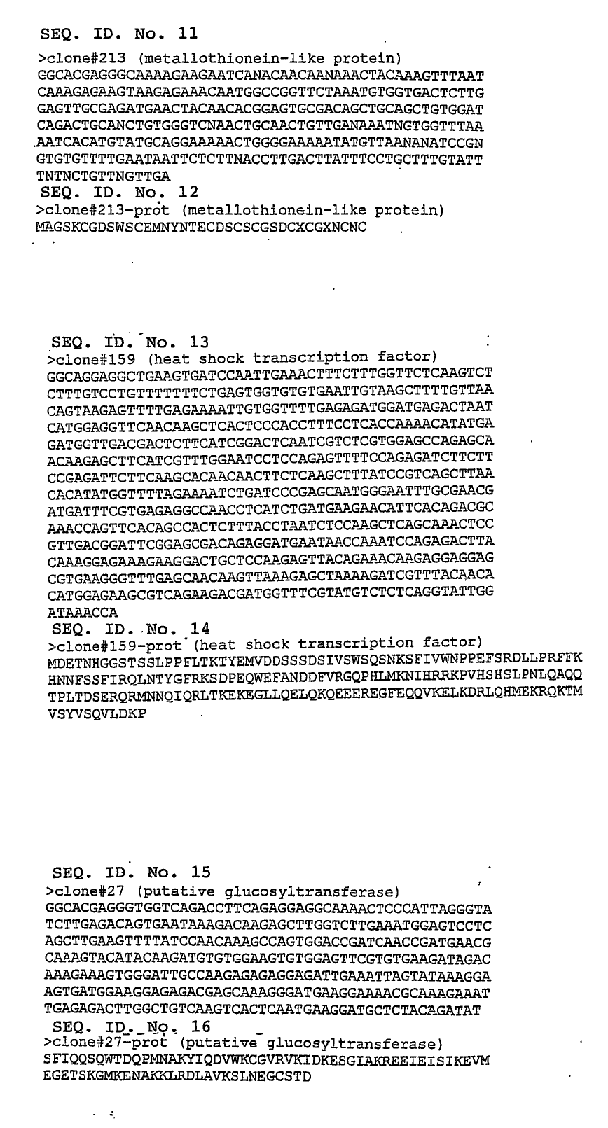

In contrast, cells expressing full-length TcHMA4 were far more sensitive to CdSO₄ than the control cells (FIG. 4 Effect of HMA4-C and HMA4 expression on cadmium tolerance in two yeast strains. Yeast BY4741 and CM100 cells transformed with the pYX212 plasmid (grey columns) and with pYX212 containing the T. caerulescens (a, b) and A. thaliana (c,d) 5′ truncated cDNA, HMA4-C (white columns), and full-length cDNA, HMA4 (black columns), were grown in liquid YNB-ura without or with 10 to 50 μM CdSO4. Cells were incubated at 30° C. for 24 h).

To investigate whether the effects of HMA4 and HMA4-C expression were strain-dependent, another wild type strain, CM100, was transformed with the recombinant pYX212-HMA4 plasmids. CM100 strain is much more sensitive to cadmium than BY4741 and cadmium tolerance of cells expressing truncated coding sequence as well as cadmium sensibility of cells expressing full-length coding sequence were confirmed in CM100 yeast strain.

To compare TcHMA4 with its Arabidopsis orthologue, a full-length AtHMA4 cDNA and its truncated version coding for the C-terminal portion (residues 767-1172) were cloned in pYX212 and expressed in yeast.

Similar phenotypes as those described for Thlaspi sequences were observed in BY4741 and in CM100.

Nevertheless, in both yeast strains, the

TcHMA4-C and AtHMA4-C peptides showed consistent differences in their ability to confer cadmium tolerance. The tolerance conferred by AtHMA4-C was lower. This difference was visible at lower concentrations in CM100 than in BY4741 (at 20 μM CdSO₄ for CM100 and at up to 50 μM CdSO₄ for BY4741 (FIG. 4).

These results were confirmed on solid medium (on 40 μM CdSO₄).

On the contrary, there was no significant difference in the enhanced cadmium sensitivity conferred by the entire plant HMA4 protein.

Expression of HMA4 in Plants:

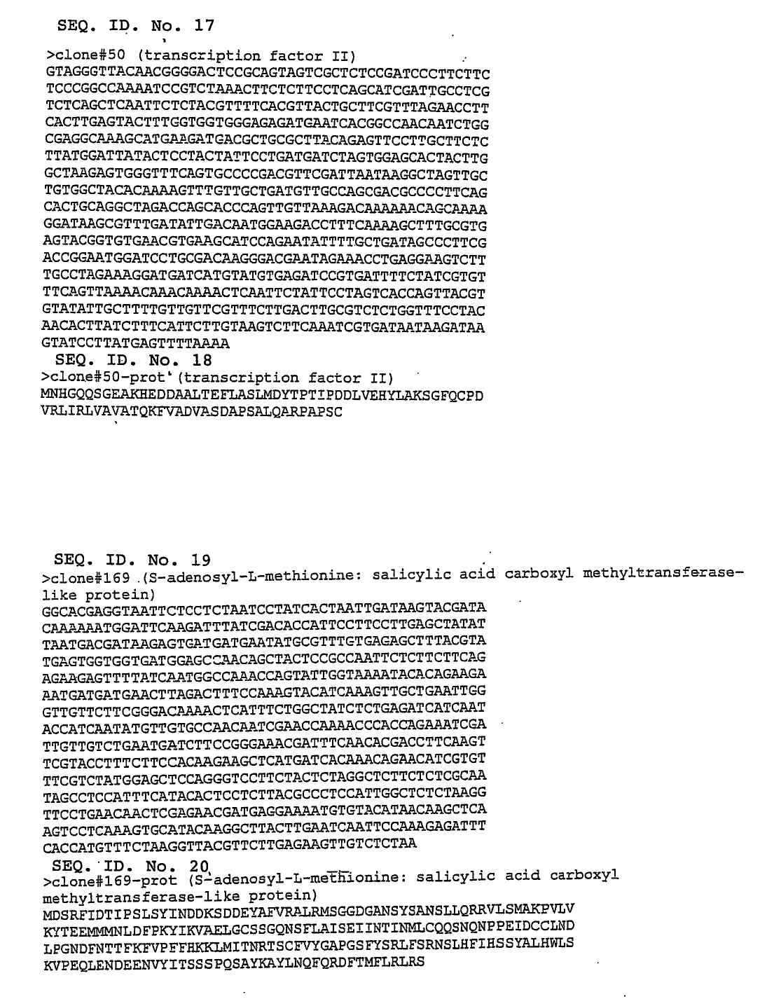

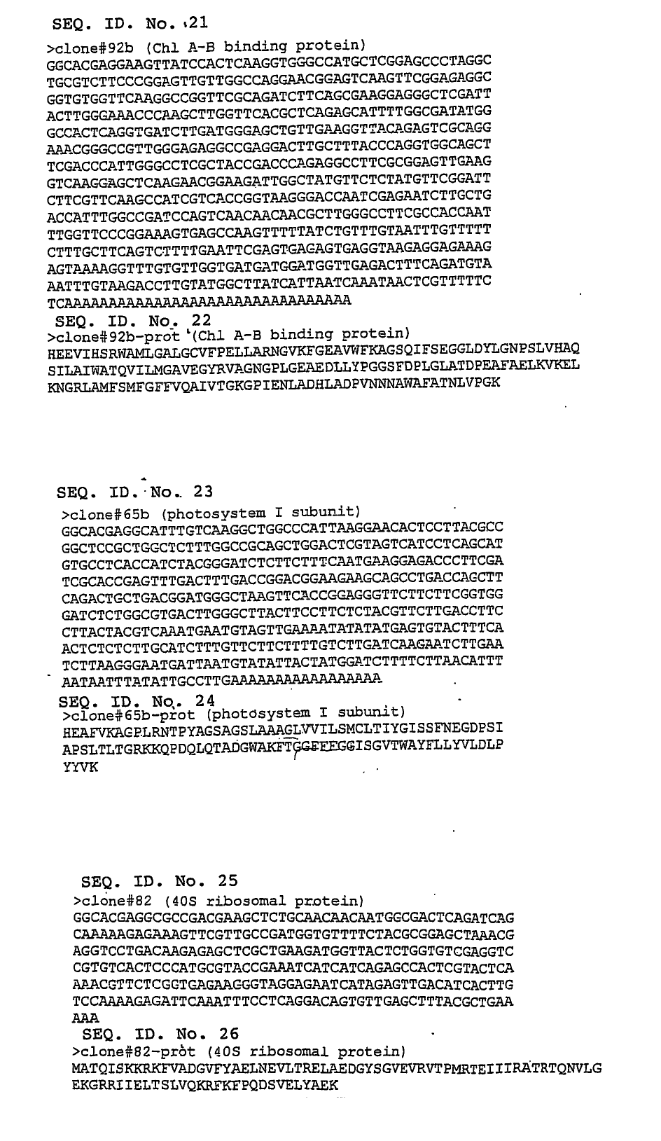

The expression of TcHMA4 was studied in planta, in shoots and roots, by Northern blot analysis under stringent conditions (FIG. 5 Northern blot of HMA4 expression in T. caerulescens and A. thaliana. (a) Total RNA was isolated from shoots and roots of the hyperaccumulator T. caerulescens and the nonaccumulator A. thaliana. Plants were exposed to 10 and 100 μM CdSO₄ for 24 h. Northern blots equally loaded with 15 μg of total RNA were probed with respectively 3′ terminal part of TcHMA4 and AtHMA4 (1.2 kb) and after stripping with 18S rRNA as a loading control. Expression levels were normalized to 18S rRNA. Results are averages (±SE) from three independent experiments. (b) Total RNA was isolated from roots of three contrasting populations of T. caerulescens different in their cadmium tolerance and accumulation: Prayon (Belgium), St Felix de Pallieres (France) and Puente Basadre (Spain). Plants were exposed to 100 μM CdSO₄ for 24 h).

In the roots of all tested 3 populations the constitutively high expression of TcHMA4 was confirmed. No significant difference in the abundance of TcHMA4 expression could be detected between these three populations by Northern blot.

Analysis of Thlaspi Caerulescens cDNAs Encoding Metallothioneins

Five different MT cDNAs have been identified. Four encoded proteins representative of the plant MT family (type-1, -2 and -3) while the fifth encoded amino acid sequence displaying similarity to invertebrate MTs but not with plant sequences. Because of the unique distribution pattern of cysteine residues in MTs, according to Cobbett and Goldsbrough (Ann. Rev. Plant Biol, Vol. 53 p 159-182)(2002), and high sequence similarity with Arabidopsis MTs, proteins-encoding Thlaspi cDNAs identified were designated as Thlaspi type-1, -2 and -3 metallothioneins (TcMTs). The cDNA encoding protein with no homology with plant proteins was named MRP, for Metallothionein Related Protein.

Type-3 Metallothioneins:

The cDNAs 10 and 51 are respectively 465 bp and 463 bp long, encoding both 67 amino acid residues. These sequences share 94% nucleic sequence identity with each other in the coding region and 92%/83% in the 3′ and 5′ untranslated regions respectively. Amino acid sequence identity was 85% and similarity 87%.

Metallothionein Related Protein (MRP):

The cDNA 114 is 626 bp long and contains a coding region of 204 bp, with a 89 bp 5′ and 300 bp 3′ untranslated regions. The open reading frame encodes a protein of 68 amino acids. Seven identical cDNA clones encoding 68 amino acid protein were isolated during the screening.

A sequence search indicates that the deduced protein has homology to invertebrate metallothioneins. No homology was found with plants. For this reason, the protein encoded by cDNA 114 was named “MRP” for Metallothionein Related Protein.

Actually, the highest homology of MRP was not found with another MT, but with ultra high sulphur keratin proteins (longer proteins) from human and mouse. However, cysteine and serine residues are responsible for this homology.

The deduced MRP sequence exhibits characteristics of MTs with regard to number of cysteine residues and molecular size, but its pattern of cysteine residues cannot be aligned with cysteines of plant MTs. MRP does not share the typical feature of plant MT proteins which are characterized by the presence of cysteine-rich domains in both N- and C-termini, with the central domain devoid of cysteines.

The arrangement of cysteine residues in MRP is peculiar. First, the 16 cysteine residues are distributed throughout the polypeptide. The two (in type 1, 2 and 3 MTs) or the three (in type 4 MTs) highly conserved cysteine-rich domains are absent. Secondly, although some cysteine residues are arranged in motifs common in plant MTs, X-Cys-Cys-X, Cys-X-Cys or single Cys residue, others appear in an atypical motif Cys₄₀-Cys-Cys.

Moreover, the deduced MRP sequence has a high serine content (19%) besides the high cysteine content (23.5%).

Cadmium Tolerance Test in Yeast:

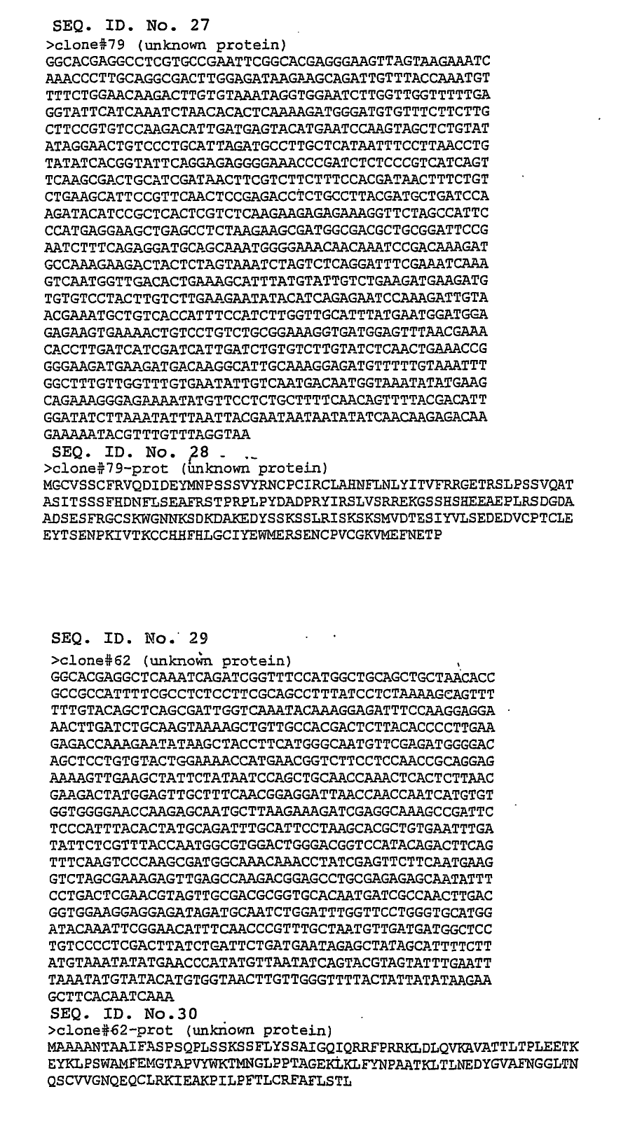

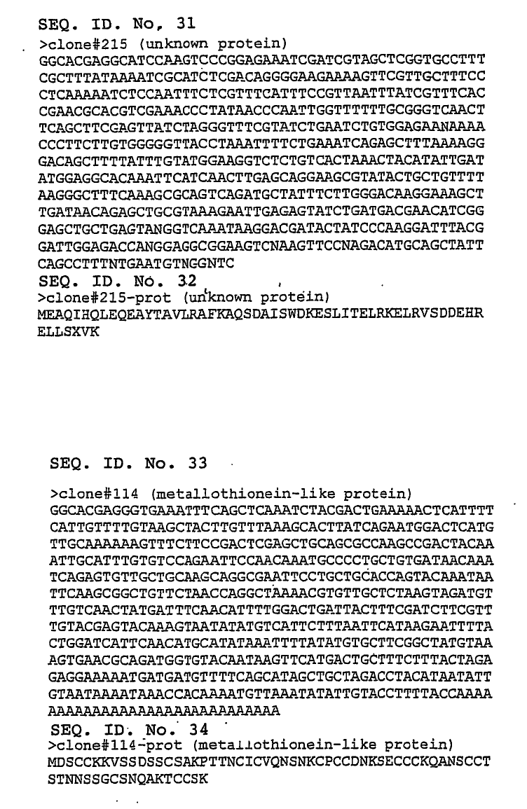

The ability of Thlaspi metallothionein cDNAs to increase cadmium tolerance of S. cerevisiae was checked using BY4741 cells expressing TcMT cDNAs for cadmium tolerance test. cDNAs expressed from pYX212 in BY4741, were used for a growth drop test on agar medium containing 0, 20 and 40 μM CdSO₄. Plasmids carrying the expression vector (pYX212) or the Thlaspi phytochelatin synthase 1 cDNA (TcPCS1) were used as negative and positive controls, respectively. Phytochelatins are known to play an important role in cadmium detoxification in plants and were previously shown to increase the cadmium tolerance in S. cerevisiae (FIG. 6: Transformants of the yeast strain BY4741 containing empty plasmid pYX212 as negative control and TcPCS as a positive control, and pYX212 with Thlapsi cDNAs of interest: TcMT3a, TcMT3b, TcMT2, TcMT1, MRP, were grown in liquid minimal medium overnight. Cultures were adjusted to A₆₀₀ of 1 and serially 10-fold diluted in water. 5 μl aliquots of each dilution were spotted either on non-selective cadmium plates or on plates with 20 and 40 μM CdSO₄. After three days of incubation at 30° C., plates were photographed. Dilutions are indicated above the figures. Two individual clones of each yeast transformants were analysed).

Cells carrying the expression vector grew normally in the absence of cadmium but were highly sensitive to cadmium and no growth was observed on 40 μM CdSO₄.

In contrast, cells expressing TcPCS1 were able to grow on 20 and 40 μM CdSO₄.

TcMT3a, TcMT3b, TcMT2 and TcMT1 cDNAs improved cadmium tolerance to the same extent, colony growth was observed at all dilutions on 20 μM CdSO₄. Cells expressing MRP showed the best cadmium tolerance and were still able to grow on 40 μM CdSO₄ at the highest dilution.

TcMT mRNA Expression in Plants:

Expression of TCMT was analysed in three contrasting populations of T. caerulescens, namely Prayon (moderately Cd tolerant with the lowest Cd concentration), Puente Basadre (the least tolerant population) and St Felix de Pallières (the most tolerant population).

RNA was isolated from three weeks old plants grown in normal medium or treated with 100 μM CdSO₄ for 72 h. The full length labelled cDNA of Thlaspi MTs were used as probes in northern blotting.

The level of TcMT3 transcripts was more abundant in shoots than in roots of Thlaspi plants and was not cadmium regulated. Abundance of TcMT3 transcripts was remarkably higher in shoots of St Felix de Pallières, the best Cd tolerant and hyperaccumulator population, than in those from Puente Basadre and Prayon. No difference between populations was observed in roots.

No difference in the level of TcMT-2 and -2 expression was found upon cadmium treatment whatever the population studied. However marked differences were observed between shoots and roots. TcMT1 mRNA was abundant in shoots and undetectable in roots whereas TcMT2 was expressed in both shoots and roots with mRNA level slightly higher in shoots than in roots.

Transformation Experiments in Non Hyperaccumulator Plants (for Example Tobacco Plants or A. thaliana Plants):

Maximum 4 genes of Thlaspi caerulescens related to cadmium tolerance will be selected and constructions in binary vectors will be made in order to overexpress them in cadmium sensitive and non hyperaccumulator plants like Arabidopsis thaliana or Tobacco plants. Control plants will be transformed with empty binary vectors (for example pBIN19).

The interest for tobacco plants comes from the fact that tobacco has no wild relatives in the European flora and the use of sterile transgenic tobacco plants is already a strategy selected by pharmaceutical firms to overproduce therapeutic molecules in fields (Queyrel, 2002). The transformation of chloroplasts or another cell compartment may be used to avoid gene flow.

Concerning the obtention and selection of transgenic lines, integration of transgenes will be tested by PCR. Overexpression will be analysed by Northern blotting, the number of transgene copy will be estimated by segregation analysis and Southern blotting. Homozygous lines with 1, maximum 2 copies will be selected among the best overexpressors since transgene stability is favoured by low copy number. Minimum 4 independent transgenic lines per construction will be selected for further study.

Concerning the characterisation of transgenic lines, a growth test in hydroponic and mineral analysis will be done as follows: seeds of selected lines will be sown and plants will be transferred in hydroponic culture where the metal treatment can be precisely and homogeneously controlled and roots as well as the leaves can be easily harvested. Fresh and dry weight of heavy metals-treated and non-treated plants will be measured. Heavy metals contents and allocation (proportion in leaves and roots) will be analysed by atomic absorption spectrophotometry. Phytoextraction capacities of the different lines (measured as the heavy metal concentration in the shoot multiplied by the shoot biomass) will be compared with the control plants and with the original hyperaccumulator species.

The best transgenic lines can be further tested on polluted soils. In the future, the best lines can be crossed to ameliorate the phytoextraction capacity.

Maximum 4 genes will be selected and constructions in binary vectors will be made in order to overexpress them in cadmium sensitive and non hyperaccumulator plants like Arabidopsis thaliana or Tobacco plants. Control plants will be transformed with empty binary vectors (for example pBIN19).

The interest for tobacco plants comes from the fact that tobacco has no wild relatives in the European flora and the use of sterile transgenic tobacco plants is already a strategy selected by pharmaceutical firms to overproduce therapeutic molecules in fields (Queyrel, 2002). The transformation of chloroplasts or another cell compartment may be used to avoid gene flow.

Concerning the obtention and selection of transgenic lines, integration of transgenes will be tested by PCR. Overexpression will be analysed by Northern blotting, the number of transgene copy will be estimated by segregation analysis and Southern blotting. Homozygous lines with 1, maximum 2 copies will be selected among the best overexpressors since transgene stability is favoured by low copy number. Minimum 4 independent transgenic lines per construction will be selected for further study.

Concerning the characterisation of transgenic lines, a growth test in hydroponic and mineral analysis will be done as follows: seeds of selected lines will be sown and plants will be transferred in hydroponic culture where the metal treatment can be precisely and homogeneously controlled and roots as well as the leaves can be easily harvested. Fresh and dry weight of heavy metals-treated and non-treated plants will be measured. Heavy metals contents and allocation (proportion in leaves and roots) will be analysed by atomic absorption spectrophotometry. Phytoextraction capacities of the different lines (measured as the heavy metal concentration in the shoot multiplied by the shoot biomass) will be compared with the control plants and with the original hyperaccumulator species.

The best transgenic lines can be further tested on polluted soils. In the future, the best lines can be crossed to ameliorate the phytoextraction capacity.

TABLE 1
Summary of the identified cDNAs.

| | | | | | |
|---|---|---|---|---|---|
| cDNA | Putative function | | | | |
| No | (number of homologous cDNA isolated) | Identity (%) | Organism | # ORF (aa) | Note |

I. Metal detoxification

| | | | | | |
|---|---|---|---|---|---|
| # 8 | Phytochelatin synthase 1 (60) | 78% on 485 aa | A. thaliana | gb AAD50593 (485) | 1 |
| # 10 | Metallothionein type 3a\|(54) | 78% on 69 aa | A. thaliana | gb AAB67234 (69) | 1 |
| # 51 | Metallothionein type 3b\| | 81% on 69 aa | A. thaliana | gb AAB67234 (69) | 1 |
| # 167 | Metallothionein type 2 (2) | 91% on 81 aa | A. thaliana | sp P25860 (81) | 1 |
| # 213 | Metallothionein type 1 (1) | 69% on 45 aa | A. thaliana | sp P43392 (45) | 1 |
| # 114 | Metallothionein related protein (7) | 45% on 24 aa | Paracentrotus | sp P80367 (68) | 1 |
| | | | lividus | | |

II. Metal transport

| | | | | | |
|---|---|---|---|---|---|
| # 64 | Cd/Zn transporting P-type ATPase (1) | 79% on 259 aa | A. thaliana | sp O6447 (1172) | 4 |
| # 71 | Cd/Zn transporting P-type ATPase (1) | 38% on 414 aa | A. thaliana | sp O6447 (1172) | 3 |
| # 165 | Cd/Zn transporting P-type ATPase (1) | 37% on 191 aa | A. thaliana | sp O6447 (1172) | 4 |
| # 199 | Cd/Zn transporting P-type ATPase (1) | 44% on 333 aa | A. thaliana | sp O6447 (1172) | 4 |

III. Signalling pathway

| | | | | | |
|---|---|---|---|---|---|
| # 159 | Heat shock transcription factor (2) | 91% on 187 aa | A. thaliana | gb AAC31792 (401) | 2 |
| # 50 | General transcription factor IID (1) | 93% on 134 aa | A. thaliana | pir T05098 (134) | 1 |

IV. Others

| | | | | | |
|---|---|---|---|---|---|
| # 169 | SAM: salicylic acid carboxyl | 71% on 197 aa | A. thaliana | dbj BAB10919 (354) | 2 |
| | methyl transferase (1) | | | | |
| # 92b | Chl A-B binding protein (1) | 98% on 169 aa | A. thaliana | gi 115767 (267 aa) | 3 |
| # 82 | 40S ribosomal protein (1) | 98% on 90 aa | A. thaliana | gi 9758155 (248 aa) | 2 |
| # 65b | Photosystem I subunit (1) | 96% on 101 aa | A. thaliana | gi 7488011 (219 aa) | 3 |
| # 27 | Glycosyltransferase | 82% on 78 aa | A. thaliana | gi2281088 (449 aa) | 4 |

V. Unknown

| | | | | | |
|---|---|---|---|---|---|
| # 62 | Unknown protein (2) | 92% on 268 aa | A. thaliana | gb AAG40376 (268) | 1 |
| # 79 | Unknown protein (1) | 71% on 232 aa | A. thaliana | emb CAC01778 (232) | 1 |
| # 215 | Unknown protein (1) | 94% on 56 aa | A. thaliana | gb AAG51060 (327) | 2 |

1 - complete coding sequence cloned
2 - complete coding sequence cloned but partially sequenced
3 - truncated coding sequence cloned
4 - truncated coding sequence cloned and partially sequenced
Results of databases searches using the BLASTX (+ BEAUTY) program. The number of times that each cDNA has been identified is indicated in brackets.
Remark concerning Table 1:
(1): complete coding sequence cloned;
(2): complete coding sequence cloned but partially sequenced;
(3): troncated coding sequence cloned;
(4): troncated coding sequence cloned and partially sequenced;
(5): troncated coding sequence cloned in yeast but further completed by Rt-PCR and 5′ RACE;
clone#8 corresponds to SEQ. ID. NO. 1 and 2;
clone#71 corresponds to SEQ. ID. NO. 3 and 4;
clone#10 corresponds to SEQ. ID. NO. 5 and 6;
clone#51 corresponds to SEQ. ID. NO. 7 and 8;
clone#167 corresponds to SEQ. ID. NO. 9 and 10;
clone#114 corresponds to SEQ. ID. NO. 33 and 34;
clone#213 corresponds to SEQ. ID. NO. 11 and 12;
clone#159 corresponds to SEQ. ID. NO. 13 and 14;
clone#27 corresponds to SEQ. ID. NO. 15 and 16;
clone#50 corresponds to SEQ. ID. NO. 17 and 18;
clone#169 corresponds to SEQ. ID. NO. 19 and 20;
clone#92b corresponds to SEQ. ID. NO. 21 and 22;
clone#65b corresponds to SEQ. ID. NO. 23 and 24;
clone#82 corresponds to SEQ. ID. NO. 25 and 26;
clone#79 corresponds to SEQ. ID. NO. 27 and 28;
clone#62 corresponds to SEQ. ID. NO. 29 and 30;
clone#215 corresponds to SEQ. ID. NO. 31 and 32.

1. An isolated and purified polypeptide useful in phytoremediation, presenting more than 40% sequence identity with the sequence SEQ ID NO: 4, and/or active fragments thereof.

2. The isolated and purified polypeptide sequence according to claim 1 wherein the sequence is isolated and purified from Thlaspi caerulescens.

3. A polynucleotide sequence encoding the polypeptide sequence according to claim 1.

4. The polynucleotide sequence according to claim 3 further comprising, operably linked to it, one or more adjacent regulatory sequence(s).

5. The polynucleotide sequence according to the claim 4 which is a sequence presenting more than 40% sequence identity with SEQ ID NO: 3, and/or active fragments thereof.

6. The fragment of the polypeptide of claim 1 having an amino acid sequence starting from the amino acid 719 up to amino acid 1134 of SEQ ID NO: 4.

7. A vector comprising the polynucleotide sequence(s) according to claim 3.

8. A recombinant host cell or plant transformed by one or more polynucleotide sequence(s) according to claim 3 or the vector according to claim 7.

9. The recombinant host cell according to claim 8, which is selected from the group consisting of bacteria (E. coli) and fungi, including yeast.

10. The recombinant host cell according to claim 9, said host cell being S. cerevisiae.

11. The recombinant host cell according to claim 8, said host cell being a plant cell.

12. The recombinant host cell or plant according to claim 8, which is a cell or plant selected from the group consisting of Arabidopsis thaliana, tobacco, Brassicaceae family plant cell or plant, and Caryophyllaceae family plant cell or plant.

13. A method for the phytoremediation treatment of a medium contaminated by heavy metals said method comprising the step of cultivating upon said contaminated medium the genetically transformed plant according to claim 8.

14. The method according to the claim 13 wherein said phytoremediation is a phytoextraction treatment of the medium which comprises the step of recovering and destroying said cultivated plant and/or the step of recovering said heavy metals from said cultivated plant.

15. The polypeptide of claim 1 presenting more than 80% sequence identity with the sequence SEQ ID NO: 4 and/or an active fragment thereof.

16. The isolated and purified polypeptide sequence according to claim 1, presenting more than 90% sequence identity with the sequence SEQ ID NO: 4 and/or an active fragment thereof.

17. The polypeptide of claim 1, presenting more than 95% sequence identity with the sequence SEQ ID NO: 4 and/or an active fragment thereof.

18. The polynucleotide sequence according to claim 5, which is a sequence presenting more than 80% sequence identity with SEQ ID NO: 3, and/or an active fragment thereof.

19. The polynucleotide sequence of claim 5 presenting more than 90% sequence identity with SEQ ID NO: 3, and/or an active fragment thereof.

20. The polynucleotide sequence of claim 5 presenting more than 95% sequence identity with SEQ ID NO: 3, and/or an active fragment thereof.

21. The method of claim 13, wherein the heavy metals are cadmium heavy metal.

22. A method for the phytoremediation treatment of a medium contaminated by heavy metals said method comprising the step of cultivating upon said contaminated medium the genetically transformed plant according to claim 12.
